(19) United States
(12) Patent Application Publication
(10) Pub. No.: US 2025/0118415 A1
(43) Pub. Date: Apr. 10, 2025

(54) NUTRITIVE RECIPE ANALYSIS SYSTEM AND METHODS

(71) Applicant: WILLOW LABORATORIES, INC., Irvine, CA (US)

(72) Inventors: Gregory A. Olsen, Lake Forest, CA (US); Shadae Zamyad, Irvine, CA (US); Quan Tran, Irvine, CA (US); Jesse Chen, Foothill Ranch, CA (US); Gerry Hammarth, Irvine, CA (US)

(21) Appl. No.: 18/907,326

(22) Filed: Oct. 4, 2024

Related U.S. Application Data
(60) Provisional application No. 63/588,501, filed on Oct. 6, 2023.

(51) Int. Cl. G16H 20/60; G06Q 50/12

(52) U.S. Cl. CPC G16H 20/60; G06Q 50/12

(57) ABSTRACT

A system can process an indication of user data, wherein the user data comprises a food item including at least one recipe having one or more ingredients, and a query, wherein the query is associated with at least one ingredient, and generate, based at least in part on the query, nutrition data associated with the at least one ingredient, wherein the nutrition data comprises preparation data, sourcing data, delivery data, and a list of nutrients for the at least one ingredient. The system can further estimate, based at least in part on the nutrition data associated with the least one ingredient, a nutritional score, then compare the estimated nutritional score to a threshold, and based on the compared nutritional score, generate one or more instructions to cause an indication of the nutritional score on a user device.

### CROSS-REFERENCE TO RELATED APPLICATIONS

The present application claims priority benefit to U.S. Provisional Application No. 63/588,501, entitled “RESTAURANT RECIPE NUTRITIONAL ANALYSIS SYSTEM AND METHODS,” filed Oct. 6, 2023, which is hereby incorporated herein by reference in its entirety.

### TECHNICAL FIELD

The present disclosure generally relates to systems and techniques for determining nutritional facts for a food item. More specifically, the present disclosure relates to systems and software for providing a nutritional score to help users evaluate a food item.

### BACKGROUND

A background is provided for introductory purposes and to aid the reader in understanding the detailed description. The background should not be taken as an admission of any prior art to the claims.

Dietitians utilize food's nutritional facts when analyzing and diagnosing a patient. The USDA maintains a nutritional facts database for common food, branded food, and manufactured food. Vendors may offer comprehensive nutritional fact databases for one or more food items. Each of these options help dietitians and patients alike perform dietary analysis at home. Local restaurants are exempt from these regulations because local restaurants often times lack the financial resources to perform a comprehensive nutritional analysis of their food items. Computers can be programmed to perform calculations and operations utilizing computer-based food analysis. Various techniques have been developed to minimize the effort required by a human user in adapting and reprogramming the computer for utilizing such computer-based food analysis.

### SUMMARY

The systems, methods, and devices described herein each have several aspects, no single one of which is solely responsible for its desirable attributes. Without limiting the scope of this disclosure, several non-limiting features will now be discussed briefly.

The health of a person can hinge on a variety of behavioral, genetic, social, dietary and other factors. In particular, behavioral and social factors such as diet, exercise, sleep, and stress are significant contributors to the development of so-called lifestyle diseases, such as heart disease, stroke, and type 2 diabetes. To persons at risk for developing such diseases, the general notion of reducing their risk through a regimen of stress reduction, sleeping more, eating better and exercising regularly may be known and understood. However, compliance with such a regimen is difficult. While there are programs that may aid a person with following such a regimen, they typically only address a limited number of factors that contribute to a person's health, especially with regards to obtaining accurate nutritional information. Further, dietitians, healthcare professionals, chefs, and/or the like attempting to create a recipe (e.g., food item) to improve a person's health lack the information required to adjust a recipe to improve the nutritional value, while minimizing economic impact and mitigating bottle-necks associated with ingredient supply chains. For example, obtaining accurate nutritional information for food items can be challenging for various reasons.

A primary obstacle to obtaining nutritional information for food items lies in the diverse methods of food preparation and cooking techniques. The way foods are cooked and processed can significantly influence their nutrient content, making it difficult to provide consistent and precise data for various dishes. Factors such as temperature, cooking time, and the addition of ingredients during cooking all contribute to the complexity of accurately assessing the nutritional value of meals.

Another contributing factor is the considerable variation in portion sizes across different dining settings. Meals served at restaurants, cafes, or even in households can differ significantly in portion sizes, leading to variations in nutrient intake. This variability poses a challenge in obtaining consistent and reliable nutritional information across different meals and eating establishments.

Additionally, hidden ingredients and preparation techniques in restaurant dishes and processed foods further complicate the task of obtaining precise nutritional information. Many meals contain added sugars, sodium, or preservatives, which are not immediately evident to consumers. These hidden components can significantly impact the overall nutritional content of a meal, making it challenging to report accurate values without detailed knowledge of the ingredients used.

The availability and comprehensiveness of food databases also play a crucial role in providing accurate nutritional information. However, these databases may not encompass every ingredient or regional cuisine, leading to gaps in data. One of the critical inputs dietitians utilize for analysis and diagnosis is food's nutritional facts is a database maintained by the U.S. Department of Agriculture (USDA). The USDA nutritional facts database includes data for common food, branded food, and manufactured food. In addition, several vendors offer comprehensive nutritional facts databases in the market. All these facilities are helping dietitians, medical professionals, dietitians, patients, and chefs alike perform dietary analysis at home. However, restaurant food is a different story. One of the significant challenges to dietitians is the lack of nutritional facts about restaurant food. USDA only requires large restaurant chains to disclose the nutritional facts of their food items. Local restaurants do not have the resources to hire professionals to perform nutritional analyses on their food items. As a result, only less than 10% of restaurants in the US disclose nutritional facts of the food they serve to the public. Dietitians, medical professionals, chefs, patients, and/or the like, must guestimate when logging food in restaurants.

Obtaining accurate nutritional information for meals is a complex task influenced by factors such as food preparation, portion sizes, limitations in food databases, hidden ingredients, and the lack of standardization in labeling and data collection. A lack of dietary information significantly affects the accuracy of the dietary analysis and thus, may put consumers at risk of obesity, allergy or another ailment. In addition certain individuals such as those with Type 1 diabetes may be at risk of poor glycemic levels because of incorrectly estimate

Disclosed herein is a nutritional analysis system (hereinafter “the system”) and methods for determining dietary data based on a food item. The system can operate on a wearable device, mobile device, desktop computer, or server running on the Internet. The system can be based on the organized aggregation of data for each ingredient applied to a food item.

The system may receive, from one or more user device(s), food data including images, text and/or another form of input including a meal, ingredients, preparation, delivery, sourcing, portion(s), a recipe, and/or the like. Along with food data, the system can further receive user data via a user input. User data can include data associated with food data, including requesting a query and/or instructing the system to tailor the system's output based on received criteria. Some example instructions associated with user data can include determining an individual meal's nutritional score or an average nutritional score for a general population, determining a general nutritional score and/or a custom score based on dietary needs (interchangeably called “dietary motivations”), such but not limited to, diabetes prevention, allergy limitations, muscle/body building, reduced sodium intake, calorie targets, portion control, increasing iron-rich foods, optimize vitamin D intake, enhance immune system function, increase energy levels, lower blood pressure, reduce risk of obesity, and/or the like. Further, user data can include instructions and data associated with determining an economic impact of a determined food item and/or generating recommendations for cost savings associated with a food item. The system can determine nutritional information based on the food data, user data, and/or nutrition data. Nutrition data can be received from user and/or retrieved based on a query. The system can query an internal and/or external database to obtain nutrition data associated with the received ingredients. For example, the system can determine specifics on nutrients, ingredients, preparation, delivery, sourcing, and/or the like of one or more food items.

The system can analyze nutritional information and generate for example, a fact summary, a nutrition based learning exercise (which can sometimes referred to herein as “nutricise” or “nutu score”), one or more recommendations regarding the food data, and/or a nutrition score (hereinafter “dietary data”). The nutritional score can be a key performance indicator of a food item. In some implementations, the nutritional score can help medical professionals, dietitians, chefs, patients, and/or the like (herein after “users”), evaluate food data. Further, the system can determine a nutritional score associated with user data. For example, nutritional scores can be generalized for entire populations and/or specific to certain desired outcomes of specific dietary needs, such but not limited to diabetes prevention, allergy limitations, muscle/body building, reduced sodium intake, calorie targets, portion control, increasing iron-rich foods, optimize vitamin D intake, enhance immune system function, increase energy levels, lower blood pressure, reduce risk of obesity, and/or the like. Advantageously, the nutritional score can be used by users to make informed adjustments to one or more food items. For example, after a user has made an adjustment to a food item, the user may transmit the adjusted food item back into the system, where the system can determine one or more outputs, such as a new nutritional score based on the adjusted food data (e.g. adjusting a food item and/or the like).

Moreover, the system can evaluate the dietary data and then generate one or more recommendations based on user data, to help users improve the food items' dietary value. Advantageously, the system can use user data to assist users to develop special food items to meet dietary needs (e.g., dietary motivation) such as to improve response to chronic diseases and/or any other need such as but not limited to diabetes prevention, allergy limitations, muscle/body building, reduced sodium intake, calorie targets, portion control, increasing iron-rich foods, optimize vitamin D intake, enhance immune system function, increase energy levels, lower blood pressure, reduce risk of obesity, and/or the like.

Additionally, the system can calculate real time the economic impact based on a user's decision to adjust a food item. The system can use dietary data along with one or more databases to determine the economic impact of adjusting a food item according to, for example, user data, food data, nutrition data, and/or any other data). Further, the system can determine impacts to one or more affected supply chains based on an adjusted food item, and instruct for example, an external system to purchase one or more ingredients based on the affects to the supply chain.

Furthermore, the system can provide an interactive user interface. The system can display via a graphical user interface (“GUI”), food data, user data, and/or nutrition data, and/or dietary data via one or more nutritional facts.

In some aspects, the techniques described herein relate to a system for estimating a nutritional score for a food item, the system including: memory that stores computer-executable instructions; and a processor in communication with the memory, wherein the computer-executable instructions, when executed by the processor, cause the processor to: process an indication of user data, wherein the user data includes a food item including at least one recipe having one or more ingredients and a query, wherein the query is associated with at least one ingredient; generate, based at least in part on the query, nutrition data associated with the at least one ingredient, wherein the nutrition data includes preparation data, sourcing data, delivery data, and/or a list of nutrients for the at least one ingredient; estimate, based at least in part on the nutrition data associated with the least one ingredient, a nutritional score; compare the estimated nutritional score to a threshold; based on the compared nutritional score, generate one or more instructions to cause an indication of the nutritional score on a user device; and transmit the generated one or more instructions to the user device.

In some aspects, the techniques described herein relate to a system, wherein the user data further includes of a dietary motivation data, and wherein the computer-executable instructions, when executed, further cause the processor to: estimate, based at least in part on the nutrition data associated with the at least one ingredient and the dietary motivation data, the nutritional score; based on the dietary motivation data, determine a threshold; compare the estimated nutritional score to a threshold; generate one or more instructions based on the nutritional score, or the dietary motivation data; and transmit the generated one or more instructions to cause an indication of the nutritional score on a user device.

In some aspects, the techniques described herein relate to a system, wherein the computer-executable instructions, when executed, further cause the processor to: based on the compared nutritional score, generate an instruction to add one or more ingredients to a shopping cart; and transmit the generated instruction to add one or more ingredients to the shopping cart, to cause an external system to purchase at least one of the one or more ingredients.

In some aspects, the techniques described herein relate to a system, wherein the computer-executable instructions, when executed, further cause the processor to: based on the compared nutritional score, generate an instruction to revise a menu; and transmit the generated instruction to cause an external system to revise a menu.

In some aspects, the techniques described herein relate to a system, wherein the computer-executable instructions, when executed, further cause the processor to: based on the compared nutritional score, generate an instruction to add one or more food items to a menu; and transmit the generated instruction, to cause an external system to add one or more food items to a menu.

In some aspects, the techniques described herein relate to a system, wherein the computer-executable instructions, when executed, further cause the processor to: based on the compared nutritional score, generate a nudge via a user device, indicating that the food item meets or does not meet at least one dietary motivation; and transmit the generated nudge, to cause a user device to indicate that a determined food item meets or does not meet a dietary motivation.

In some aspects, the techniques described herein relate to a system, wherein the computer-executable instructions, when executed, further cause the processor to: process an indication of a second food item, wherein the second food item includes at least one recipe having one or more ingredients; generate, based at least in part on the query, a second nutrition data associated with the at least one ingredient of the second food item, wherein the nutrition data includes of preparation data, sourcing data, delivery data, and a list of nutrients for the at least one ingredient of the second food item; estimate, based at least in part of the nutrition data associated with the at least one ingredient of the second food item, a second nutrition score; compare the nutrition score and the second nutrition score; and based on the compared nutrition score and the second nutrition score, transmit one or more instructions to cause an indication of the compared nutritional scores on a user device.

In some aspects, the techniques described herein relate to a system, wherein the user data further includes a dietary motivation, and wherein the computer-executable instructions, when executed, further cause the processor to: estimate, based at least in part on the second nutrition data and the dietary motivation data, a second nutritional score; based on the dietary motivation data, determine a threshold; compare the estimated second nutritional score to a threshold; generate one or more instructions based on the second nutritional score, or the dietary motivation data; and transmit the generated one or more instructions to further cause an indication of the second nutritional score on a user device.

In some aspects, the techniques described herein relate to a system, wherein the motivation data further includes an instruction to generate the nutritional score according to a user or a general population.

In some aspects, the techniques described herein relate to a system, wherein the dietary motivation data includes of a threshold wherein the threshold is based at least in part on a nutritional analysis for diabetes prevention, allergy limitations, muscle/body building, reduced sodium intake, calorie targets, portion control, increasing iron-rich foods, optimize vitamin D intake, enhance immune system function, increase energy levels, lower blood pressure, or reduce risk of obesity.

In some aspects, the techniques described herein relate to a system, wherein the list of nutrients includes calories, carbohydrates, sugars, dietary fiber, protein, saturated fat, monounsaturated fat, polyunsaturated fat, cholesterol, sodium, potassium, calcium, iron, or Vitamins.

In some aspects, the techniques described herein relate to a system, wherein the computer-executable instructions, when executed, further cause the processor to: estimate a fact summary based at least in part on the nutrition data associated with the at least one ingredient, wherein the fact summary includes a name of a recipe, a description of the preparation, delivery, and sourcing of the at least one ingredient, and a list of nutrients associated with the at least one ingredient.

In some aspects, the techniques described herein relate to a system, wherein the computer-executable instructions, when executed, further cause the processor to: estimate, based at least in part on the nutrition data associated with the at least one ingredient, a nutricise, wherein the nutricise includes a learning activity that can help users understand healthy eating habits that incorporates physical fitness into the activity.

In some aspects, the techniques described herein relate to a system, wherein the computer-executable instructions, when executed, further cause the processor to estimate an economic impact based on the nutrition data.

In some aspects, the techniques described herein relate to a system including: one or more computer-readable storage mediums having program instructions embodied therewith; and one or more processors configured to execute the program instructions to cause the system to perform the computer-implemented method.

In some aspects, the techniques described herein relate to a computer program product including one or more computer-readable storage mediums having program instructions embodied therewith, the program instructions executable by one or more processors to cause the one or more processors to perform the computer-implemented method.

In some aspects, the techniques described herein relate to a system as described herein.

In some aspects, the techniques described herein relate to a computer-implemented method as described herein. According to various implementations, large amounts of data are automatically and dynamically calculated interactively in response to user inputs, and the calculated data is efficiently and compactly presented to a user by the system. Thus, in some implementations, the user interfaces described herein are more efficient as compared to previous user interfaces in which data is not dynamically updated and compactly and efficiently presented to the user in response to interactive inputs.

Further, as described herein, the system may be configured and/or designed to generate user interface data useable for rendering the various interactive user interfaces described. The user interface data may be used by the system, and/or another computer system, device, and/or software program (for example, a browser program), to render the interactive user interfaces. The interactive user interfaces may be displayed on, for example, electronic displays (including, for example, touch-enabled displays).

Additionally, it has been noted that design of computer user interfaces that are useable and easily learned by humans is a non-trivial problem for software developers. The present disclosure describes various implementations of interactive and dynamic user interfaces that are the result of significant development. This non-trivial development has resulted in the user interfaces described herein which may provide significant cognitive and ergonomic efficiencies and advantages over previous systems. The interactive and dynamic user interfaces include improved human-computer interactions that may provide reduced mental workloads, improved decision-making, reduced work stress, and/or the like, for a user. For example, user interaction with the interactive user interface via the inputs described herein may provide an optimized display of, and interaction with, models and model-related data, and may enable a user to more quickly and accurately access, navigate, assess, and digest the model-related data than previous systems.

Further, the interactive and dynamic user interfaces described herein are enabled by innovations in efficient interactions between the user interfaces and underlying systems and components. For example, disclosed herein are improved methods of receiving user inputs (including methods of interacting with, managing, and minimizing the average number of presses for a user to log an action), translation and delivery of those inputs to various system components, automatic and dynamic execution of complex processes in response to the input delivery, automatic interaction among various components and processes of the system, and automatic and dynamic updating of the user interfaces (to, for example, display the health-related data). The interactions and presentation of data via the interactive user interfaces described herein may accordingly provide cognitive and ergonomic efficiencies, among various additional technical advantages over previous systems.

Thus, various implementations of the present disclosure can provide improvements to various technologies and technological fields, and practical applications of various technological features and advancements. For example, as described above, existing computer-based modeling technology is limited in various ways, and various implementations of the disclosure provide significant technical improvements over such technology. Additionally, various implementations of the present disclosure are inextricably tied to computer technology. In particular, various implementations rely on operation of technical computer systems and electronic data stores, automatic processing of electronic data, and the like. Such features and others (e.g., processing and analysis of large amounts of electronic data, management of data migrations and integrations, and/or the like) are intimately tied to, and enabled by, computer technology, and would not exist except for computer technology. For example, the interactions with, and management of, computer-based health data described below in reference to various implementations cannot reasonably be performed by humans alone, without the computer technology upon which they are implemented. Further, the implementation of the various implementations of the present disclosure via computer technology enables many of the advantages described herein, including more efficient management of various types of electronic data (including computer-based health data).

Various combinations of the above and below recited features, embodiments, implementations, and aspects are also disclosed and contemplated by the present disclosure.

Additional implementations of the disclosure are described below in reference to the appended claims, which may serve as an additional summary of the disclosure.

In various implementations, systems and/or computer systems are disclosed that comprise a computer-readable storage medium having program instructions embodied therewith, and one or more processors configured to execute the program instructions to cause the systems and/or computer systems to perform operations comprising one or more aspects of the above-and/or below-described implementations (including one or more aspects of the appended claims).

In various implementations, computer-implemented methods are disclosed in which, by one or more processors executing program instructions, one or more aspects of the above- and/or below-described implementations (including one or more aspects of the appended claims) are implemented and/or performed.

In various implementations, computer program products comprising a computer-readable storage medium are disclosed, wherein the computer-readable storage medium has program instructions embodied therewith, the program instructions executable by one or more processors to cause the one or more processors to perform operations comprising one or more aspects of the above- and/or below-described implementations (including one or more aspects of the appended claims).

### BRIEF DESCRIPTION OF DRAWINGS

Throughout the drawings, reference numbers may be re-used to indicate correspondence between referenced elements. The drawings are provided to illustrate example embodiments described herein and are not intended to limit the scope of the disclosure.

FIG. 1 is a block diagram of an illustrative operating environment of a nutritional analysis system according.

FIG. 2 is a flow diagram illustrating the operations performed by the components of the operating environment to determine a nutritional score.

FIG. 3 is a flow diagram depicting an example, routine to generate instructions based on a nutritional score.

FIG. 4 is an additional example operational environment for a nutritional analysis system.

FIG. 5 is an example computing environment that may implement one or more aspects of the modules and/or functionality described herein.

FIG. 6 illustrates an example disease management system that may be part of a disease management environment or used as an interleaved device.

FIG. 7 illustrates an example implementation of a disease management system.

### DETAILED DESCRIPTION

Although certain preferred implementations, embodiments, and examples are disclosed below, the inventive subject matter extends beyond the specifically disclosed implementations to other alternative implementations and/or uses and to modifications and equivalents thereof. Thus, the scope of the claims appended hereto is not limited by any of the particular implementations described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain implementations; however, the order of description should not be construed to imply that these operations are order dependent. Additionally, the structures, systems, and/or devices described herein may be embodied as integrated components or as separate components. For purposes of comparing various implementations, certain aspects and advantages of these implementations are described. Not necessarily all such aspects or advantages are achieved by any particular implementation. Thus, for example, various implementations may be carried out in a manner that achieves or optimizes one advantage or group of advantages as taught herein without necessarily achieving other aspects or advantages as may also be taught or suggested herein.

### Overview

Improvement of overall health through a holistic approach can be a complicated endeavor. Numerous dietary, activity, and personal health factors can be considered. The data associated with such factors can be numerous and difficult to parse down to actionable instruction (e.g., for an end user). As described above, disclosed herein is a nutritional analysis system (hereinafter “the system”) and methods for determining dietary data associated with a food item. The system can operate on a wearable device, mobile device, desktop computer, or server running on the Internet. The system can be based on the organized aggregation of data for each ingredient applied to a food item.

The system may receive, from one or more user device(s), food data including images, text and/or another form of input, including a meal, nutrients, ingredients, preparation, delivery, sourcing, portion(s), a recipe, and/or the like. Along with food data, the system can further receive user data via a user input. User data can include data associated with food data, including requesting a query and/or instructing the system to tailor the system's output based on received criteria. Some example instructions associated with user data can include determining an individual meal's nutritional score or an average nutritional score for a general population, determining a general nutritional score and/or a custom nutritional score based on dietary needs, such but not limited to, diabetes prevention, allergy limitations, muscle/body building, reduced sodium intake, calorie targets, portion control, increasing iron-rich foods, optimize vitamin D intake, enhance immune system function, increase energy levels, lower blood pressure, reduce risk of obesity, and/or the like. Further, user data can include instructions and data associated with determining an economic impact of a determined food item and/or generating recommendations for cost savings associated with a food item. The system can determine nutritional information based on the food data, user data, and/or nutrition data. Nutrition data can be received from user and/or retrieved based on a query. The system can query an internal and/or external database to obtain nutrition data associated with the received food data and/or user data. For example, the system can determine specifics on nutrients, ingredients, preparation, delivery, sourcing, and/or the like of one or more food items.

The system can analyze nutritional information to generate for example, a fact summary, a nutricise, one or more recommendations regarding the food data, and/or a nutrition score (hereinafter “dietary data”). The nutritional score can be a key performance indicator of a food item. In some implementations, the nutritional score can help medical professionals, dietitians, chefs, patients, and/or the like (herein after “users”), evaluate food data. For example, a nutritional score can be compared to a threshold value, to determine whether the food item meets criteria associated with one or more dietary motivations. Further, the system can determine a nutritional score associated with user data. For example, nutritional scores can be generalized for entire populations and/or specific to certain desired outcomes of specific dietary needs, such but not limited to diabetes prevention, allergy limitations, muscle/body building, reduced sodium intake, calorie targets, portion control, increasing iron-rich foods, optimize vitamin D intake, enhance immune system function, increase energy levels, lower blood pressure, reduce risk of obesity, and/or the like. Advantageously, the nutritional score can be used by users to make informed adjustments to one or more food items. For example, after a user has made an adjustment to a food item, the system may receive the adjusted food item and determine one or more outputs, such as a second nutritional score based on the adjusted food data. The second nutritional score can be displayed by the system to a user, such that a user may see differences in the nutritional score (e.g., nutritional and/or economic outcomes of one or more adjustments to a recipe).

Moreover, the system can evaluate the dietary data and generate one or more recommendations based on, for example, user data, to help users improve the food item's dietary value. Advantageously, the system can use user data to assist users to develop special food items to meet individualized dietary needs (e.g., dietary motivation) such as to improve response to chronic diseases and/or any other needs such as but not limited to diabetes prevention, allergy limitations, muscle/body building, reduced sodium intake, calorie targets, portion control, increasing iron-rich foods, optimize vitamin D intake, enhance immune system function, increase energy levels, lower blood pressure, reduce risk of obesity, and/or the like.

Additionally, the system can calculate real time the economic impact based on a user's decision to adjust a food item. The system can use dietary data along with one or more databases to determine the economic impact of adjusting a food item according to, for example, user data (or any other data). Further, the system can determine impacts to one or more affected supply chains based on an adjusted food item, and instruct for example, an external system to purchase one or more ingredients based on the affects to the supply chain.

Furthermore, the system can provide an interactive user interface. The system can display via a GUI, food data, user data, nutrition data, and/or dietary data. Further, the system can generate instructions and/or recommendations, and transmit the instructions and/or recommendations to a user device, to cause the user device to display the one or more recommendations. Such recommendations may be based on the numerous different factors discussed herein weighted together using a modelling framework. Advantageously, the system can generate a simple nutritional score and/or instructions for the user based on the numerous factors. In this way, numerous different factors can be condensed into a straightforward user display.

### Example Operational Environment of a Nutrition Analysis System

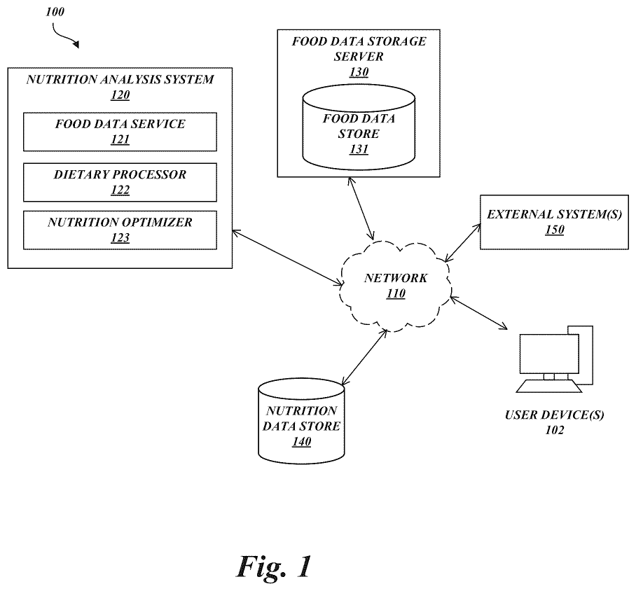

FIG. 1 is a block diagram of an illustrative operating environment 100 in which a nutritional analysis system 120 (hereafter “system 120”) uses food data storage server 130 and nutrition data store 140 to determine and display dietary data and recommendation(s) to user device(s) 102 as well as transmit one or more instructions based on the dietary data and/or recommendation(s) to external system(s) 150 via network 110 of a user 101. Further, the operating environment 100 includes food data storage server 130 used to transmit and/or receive nutrition data stored in food data store 131 to and/or from the system 120. In addition, the operating environment 100 includes user device(s) 102 that can communicate, for example, a food item to the system 120, external system(s) 150 and/or food data storage server 130.

The system 120 can be a computing system configured to determine dietary data, generate instruction(s), and/or transmit recommendation(s) to, for example, external system(s) 150, user device(s) 102, and/or food data storage server 130. For example, the system 120 can obtain food data, a food item, nutrition data, and user data from user device(s) 102 to determine dietary data including a fact summary, a nutricise, one or more recommendations regarding the food data, a nutritional score, and/or instructions for external system(s) 150 to execute a purchase and/or change a food item according to the dietary data.

The system 120 may be a single computing device, or it may include multiple distinct computing devices, such as computer servers, logically or physically grouped together to collectively operate as a server system. The components of the system 120 can be implemented in application-specific hardware (e.g., a server computing device with one or more ASICs) such that no software is necessary, or as a combination of hardware and software. In addition, the modules and components of the system 120 can be combined on one server computing device or separated individually or into groups on several server computing devices. In some implementations, the system 120 may include additional or fewer components than illustrated in FIG. 1. The system 120 can include various modules, components, data stores, and/or the like to execute dietary analysis and instructional functionality described herein. For example, the system 120 can include a food data service 121, a dietary processor 122, and/or a nutrition optimizer 123.

The food data service 121 can query and select nutrition data based on food data and/or user data. For example, food data service 121 can receive, from food data storage server 130, user device(s) 102, and/or external system(s) 150, food data and/or user data. Food data can include images, text and/or another form of data including for example, a meal, nutrients, ingredients, preparation, delivery, sourcing, portion(s), a food item, and/or the like. User data can include, for example, data associated with food data, including requesting a query and/or instructing the system 120 to tailor the system's output based on received criteria. Some example instructions associated with user data can include a request to determine a nutritional score based on an individual and/or based on the general population, a request to determine a general nutritional score and/or a custom score based on dietary needs, such but not limited to, diabetes prevention, allergy limitations, muscle/body building, reduced sodium intake, calorie targets, portion control, increasing iron-rich foods, optimize vitamin D intake, enhance immune system function, increase energy levels, lower blood pressure, reduce risk of obesity, and/or the like. Further, user data can include instructions associated with a request to determine an economic impact and/or instructions to generate recommendations to change one or more ingredients based on for example, a target cost savings and/or a dietary need associated with a food item. Additionally and/or alternatively, user data and/or food data as described herein can be interchangeably called and/or combined into “user data”. In various implementations, user data includes food data, and may be received by the food data service 121.

In some examples, the user device can send a photo of food to the food data service 121. The food data service 121 can use artificial intelligence (e.g., computer visions) to determine the type and/or quantity of food from the photo. The type and/or quantity of food can be included in food data and may be used for estimating a nutritional score.

The food data service 121 can analyze the received user data and/or food data, and extract food items, nutrients, ingredients, preparation, delivery, sourcing data, portioning data, and/or the like. Additionally, food data service 121 may determine one or more recipes for the extracted food item. Further, based on the determined recipe and/or the food data, the food data service 121 can query a data base to generate nutrition data. As an illustrative example, food data service 121 can access and retrieve nutrition data based on one or more determined food items from nutrition data store 140. Nutrition data can include data associated with one or more nutrients, ingredients, preparation, delivery, sourcing, and/or the like for a food item. As an example, nutrition data associated with user data and/or food data can include but is not limited to: calories, carbohydrates, sugars, dietary fiber, protein, saturated fat, monounsaturated fat, polyunsaturated fat, cholesterol, sodium, potassium, calcium, iron, Vitamins (e.g., C, A, E). Nutrition data can be associated with a food item, a recipe, an ingredient, and/or the like. Further, food data service 121 may calculate and/or query for nutrition data for each ingredient of a recipe based on one or more aspects such as for example preparation, cooking time, servings yield, storage and/or reheating, as part of a food item and/or recipe, and/or the like. The food data service 121 can query a data base for nutrition data based for a specific recipe and/or query a database for several recipes associated with the user data and/or food data. For example, the food data service 121 can retrieve nutritional information for one or more recipes based on a request and/or availability of ingredients as extracted from the food data, and generate a table including, for example, nutrition data associated with preparation, sourcing, delivery, and/or one or more nutrients for one or more food items (e.g., such as ingredients and/or a recipe).

Additionally, food data service 121 can aggregate the received user data, food data, and/or queried nutrition data (e.g., nutritional information). The food data service 121 can further transmit the aggregated nutritional information to one or more components of the operating environment 100 such as the dietary processor 122, the nutrition optimizer 123, user device(s) 102, external system(s) 150, and/or food data storage server 130.

The dietary processor 122 can receive data from, for example, the food data service 121. Data received by the dietary processor 122 can include user data, food data, and/or nutrition data. Alternatively, dietary processor 122 can receive food data, user data, and/or nutrition data from food data storage server 130, user device(s) 102, external system(s) 150, and/or from nutrition data store 140. The dietary processor 122 can extract the received nutritional information and/or generate additional information based on the received nutritional information. For example, the dietary processor 122 can adjust and/or generate nutrition data based on, for example, food data. As an illustrative example, the dietary processor 122 can adjust the nutrition data of one or more ingredients of a recipe based the preparation and delivery of ingredients, and/or based on user data (e.g., user input indicating for example, whether the food was previously frozen, packaging, and/or a dwell time).

In some implementations, the dietary processor 122 can determine a fact summary based on the received nutritional information. A fact summary can display information based on an analysis of the user data, the food data, and the nutrition data. A fact summary can include the name of a recipe, as well as data associated with the recipe such as preparation, delivery, sourcing, and/or the like. Further, a fact summary can include for example, a list of nutrients associated with an ingredient, a recipe, a food item, and/or the like. The list of nutrients can include, but is not limited to calories, carbohydrates, sugars, dietary fiber, protein, saturated fat, monounsaturated fat, polyunsaturated fat, cholesterol, sodium, potassium, calcium, iron, Vitamins (e.g., C, A, E). The fact summary can further include serving information (e.g., 1, 2, 3, and/or more servings) per recipe along with a serving size (e.g., 1 cup, 227 g, and/or the like). The fact summary can further include a daily value (e.g., % DV) for each of the nutrients. The dietary processor 122 can transmit the fact summary to, for example, user device(s) 102, external system(s) 150, food data storage server 130 via network 110.

Additionally, the dietary processor 122 can generate a custom learning activity (e.g., a “nutricise”) based on the received user data, the food data, and/or the nutrition data. A nutricise can be a learning activity that can help users understand healthy eating habits that incorporates physical fitness into the activity. In some examples, a nutricise can be a song based about the nutritional information based on the user data, the food data, and/or the nutrition data. The dietary processor 122 can transmit the generated nutricise to, for example, user device(s) 102, external system(s) 150, food data storage server 130 via network 110.

The dietary processor 122 can further determine a nutritional score. The nutritional score can be a key performance indicator of a food item, a recipe, an ingredient, and/or the like. In some implementations, the nutritional score can help users, evaluate food data. For example, a nutritional score can be compared to a threshold value, to determine whether the food item meets, for example, one or more dietary motivations. Further, the dietary processor 122 can determine a nutritional score associated with user data received from, for example, the food data service 121, user device(s) 102, external system(s) 150, and/or the like. For example, nutritional scores can be generalized for entire populations and/or specific to certain desired outcomes of specific dietary needs, such but not limited any of the dietary needs discussed above. As an illustrative example, the dietary processor 122 can receive user data, food data, and nutrition data from the food data service 121, where the user data is associated with a user determined recipe and user determined dietary need (e.g., body building). The dietary processor 122 may, in response to the received data, calculate a nutritional score for the determined recipe based on a calculation of whether the user selected recipe meets the user's specific goals as identified by the dietary need (e.g., whether the meal meets the dietary needs specified by the user associated with body building, such as whether the meal is high in carbohydrates, protein, and/or the like). The dietary processor 122 can transmit the generated nutritional score to, for example, user device(s) 102, external system(s) 150, food data storage server 130 via network 110.

Advantageously, the nutritional score can be used to make informed adjustments to one or more food items. For example, after the dietary processor 122 has generated a nutritional score, the score may be transmitted to the user via user device(s) 102, where the user may make an adjustment to a food item. The user may transmit the adjusted food item back to the dietary processor 122, where the dietary processor 122 can determine a second nutritional score. Additionally and/or alternatively, the dietary processor 122 can determine a second fact summary, and/or a second Nutricise based on the adjusted food data (e.g. adjusting a food item, an ingredient, a recipe, and/or the like).

The dietary processor 122 can further aggregate the fact summary, the nutricise, and/or the nutrition score (e.g., dietary data). As mentioned above, the generated dietary data can be transmitted to one or more components of the operating environment 100 such as the nutrition optimizer 123, the user device(s) 102, the food data storage server 130, external system(s) 150, via network 110.

The nutrition optimizer 123 can receive dietary data from for example, the dietary processor 122. Once the nutrition optimizer 123 receives the dietary data (e.g., including the fact summary, the determined nutricise, the nutrition score) and/or the nutritional information (e.g., the food data, user data, and nutrition data), the nutrition optimizer 123 can generate recommendation(s) and/or generate instruction(s). The recommendation(s) can include an economic impact and/or one or more recommendations associated with, for example, adjusting a recipe to further optimize the recipe according to user data. The instruction(s) generated by the nutrition optimizer 123 can include computer executable instructions that may be executed by for example, external system(s) 150, in response to the analysis of dietary data and/or nutritional information.

In various implementations, the nutrition optimizer 123 can determine an economic impact based on data associated with a recipe. An economic impact can include an estimated cost associated with creating and/or adjusting a recipe. The associated cost can be relative to, for example, historical costs associated with the determined recipe as received from the food data storage server 130. Additionally and/or alternatively, historical costs can be included as part of user data transmitted to the food data service 121. Further, the nutrition optimizer 123 can transmit instructions to external system(s) 150 to execute a task associated with the determined economic impact as described herein.

Additionally and/or alternatively, the nutrition optimizer 123 may generate recommendation(s) based on the dietary data and/or the nutritional information. For example, the nutrition optimizer 123 can generate a recommendation to adjust a recipe to meet one or more user defined dietary motivation and/or to meet a user defined economic goal. As an illustrative example, nutrition optimizer 123 can receive dietary data including a user defined economic goal to generate a recipe based on a target cost savings. The nutrition optimizer 123 can, based on the received dietary data, review ingredients, nutritional value associated with nutrients, ingredients, preparation, delivery and/or other data associated with the recipe, along with user defined data, and recommend one or more cost savings initiatives based on the ingredients and/or the recipe. The recommendations can be transmitted to, for example user device(s) 102 and/or external system(s) 150.

In various implementations, the nutrition optimizer 123 can generate instruction(s) to add one or more ingredients to an acquisition list (e.g., a shopping cart) based on the results of an economic analysis for a food item. The instructions may be transmitted to external system(s) 150, to cause the external system(s) 150 to purchase ingredient(s). In various implementations, the nutrition optimizer 123 can transmit instructions to update and/or revise a menu based on the economic impact analysis. For example, external system(s) 150 can include a restaurant management system, wherein the nutrition optimizer 123 can determine an economic impact of an adjusted recipe and generate instructions to cause a restaurant management system to include one or more recipes as part of a restaurant's menu.

Additionally, the nutrition optimizer 123 can generate instructions to update and/or nudge a user as part of a diet management system. The nutrition optimizer 123 can, for example, receive dietary data from the dietary processor 122 including user data defining a user's dietary motivations. The nutrition optimizer 123 can determine for a selected recipe, one or more adjustments to the recipe to support the user's dietary motivations, and/or determine whether a recipe meets a user's dietary motivations. Alternatively, the nutrition optimizer 123 may recommend one or more alternative recipes that may meet the user's dietary motivations if the nutrition optimizer 123 determines that adjustment to the user's recipe may not meet the user dietary motivations. The nutrition optimizer 123 may further transmit a set of instructions to user device(s) 102, providing a nudge informing the user that the selected recipe meets/does not meet one or more of the user's dietary motivations.

Food data storage server 130 can be a computer system configured to store and provide access to a database of food data, nutrition data, and/or the like. For example, food data storage server 130 can obtain food data and/or user data from one or more of user device(s) 102, an external system(s) 150, the system 120, and/or generate food data and/or user data based on one or more user inputs. Additionally, the food data storage server 130 can generate new food data (e.g., new food items and/or new ingredients) based on, for example, extrapolation of nutrients, ingredients, preparation, delivery, sourcing, and/or the like for one or more food items. The food data storage server 130 can store food data and/or user data in food data store 131. Further, food data storage server 130 can receive and maintain a history of data including dietary data generated by dietary processor 122, nutritional information as generated by food data service 121, economic impact and/or recommendation data as generated by nutrition optimizer 123, and/or any other data from one or more components of the operating environment 100 such as from the user device(s) 102 and/or external system(s) 150.

The nutrition data store 140 can store nutrition data from, for example, one or more nutrition databases such as an online database (e.g., USDA nutritional database, European Food Safety Authority Comprehensive European Food Consumption Database, Canadian Nutrient File, a private nutritional database, and/or the like), a restaurant management system, shopping carts, diet management systems and/or any other system that generates nutrition data. Nutrition data included in the nutrition data store 140 can include, for example, data associated with one or more nutrients, ingredients, preparation, delivery, sourcing, and/or the like, for one or more food items. As an example, nutrition data can include calories, carbohydrates, sugars, dietary fiber, protein, saturated fat, monounsaturated fat, polyunsaturated fat, cholesterol, sodium, potassium, calcium, iron, Vitamins (e.g., C, A, E), and/or the like. Further, nutrition data for each food item can vary based on one or more aspects such as for example preparation, cooking time, servings yield, storage and/or reheating, and/or the like.

While the nutrition data store 140 is depicted as being external to the system 120, this is not meant to be limiting. For example, not shown, the nutrition data store 140 can be internal to the system 120. Additionally and/or alternatively, the nutrition data store 140 may be a database residing on user device(s) 102 that is transmitted to the system 120 via network 110. Furthermore, not shown, the functionality of the system 120 and the food data storage server 130 can be combined into the same component or entity.

External system(s) 150 can include a third-party server and/or data store implemented as a computer system having logical elements. In an implementation, the logical elements may comprise program instructions recorded on one or more machine-readable storage media. Alternatively, the logical elements may be implemented in hardware, firmware, or a combination thereof. The external system(s) 150 may include one or more of restaurant management systems, shopping carts, diet management systems and/or any other system that may receive data from, and/or transmit data to the system 120. As an illustrative example, external system(s) 150 can include a shopping cart that receives instructions from, for example, the nutrition optimizer 123 to purchase one or more ingredients based on a determined economic impact. While external system(s) 150 are depicted as being external to the system 120, this is not meant to be limiting. For example, not shown, one or more functionality of the external system(s) 150 may be included in the system 120. Additionally and/or alternatively, one or more functionality of the external system(s) 150 may be included as part of user device(s) 102, that interact with the system 120 via network 110. Furthermore, not shown, the functionality of the external system(s) 150 and the food data storage server 130 can be combined into the same component or entity.

Users may use user device(s) 102 to view and/or interact with a GUI provided by the system 120. For example, the user device(s) 102 can include a wide variety of computing devices, including personal computing devices, terminal computing devices, laptop computing devices, tablet computing devices, electronic reader devices, mobile devices (e.g., desktop computer, notebook computer, smartphone, or any other type of computing device) wearable devices (e.g., smartwatches, fitness trackers, smart rings, ECG monitors, activity sleep monitors, heart rate monitors, pulse oximeters, glucose monitors, insulin pumps, and/or the like), and associated software (e.g. a browser capable of rendering output from information provided by, for example, the system 120, food data storage server 130, and/or external system(s) 150).

The network 110 may include any wired network, wireless network, or combination thereof. For example, the network 110 may be a personal area network, local area network, wide area network, over-the-air broadcast network (e.g., for radio or television), cable network, satellite network, cellular telephone network, or combination thereof. As a further example, the network 110 may be a publicly accessible network of linked networks, possibly operated by various distinct parties, such as the Internet. In some implementations, the network 110 may be a private or semi-private network, such as a corporate or university intranet. The network 110 may include one or more wireless networks, such as a Global System for Mobile Communications (GSM) network, a Code Division Multiple Access (CDMA) network, a Long Term Evolution (LTE) network, or any other type of wireless network. The network 110 can use protocols and components for communicating via the Internet or any of the other aforementioned types of networks. For example, the protocols used by the network 110 may include Hypertext Transfer Protocol (HTTP), HTTP Secure (HTTPS), Message Queue Telemetry Transport (MQTT), Constrained Application Protocol (CoAP), and the like. Protocols and components for communicating via the Internet or any of the other aforementioned types of communication networks are well known to those skilled in the art and, thus, are not described in more detail herein.

### Example Flow Diagram for Determining Nutritional, Dietary, and Optimized Data

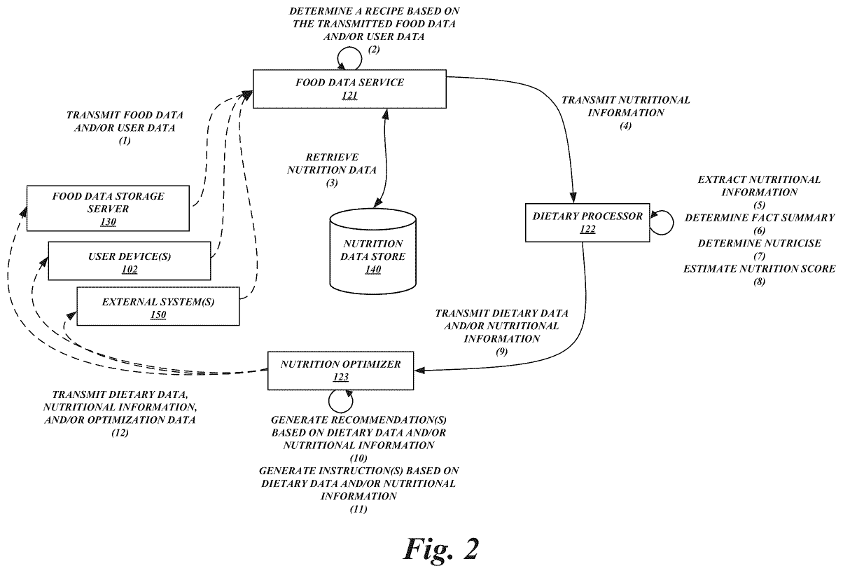

FIG. 2 is a flow diagram illustrating the operations performed by the components of the operating environment 100 of FIG. 1 to conduct a nutritional (or nutritive) analysis based on user data and/or external data from, for example a server.

As illustrated in FIG. 2, food data storage server 130, user device(s) 102, and/or external system(s) 150 can transmit food data (e.g., images, text and/or another form of data, including among other things, a meal, nutrients, ingredients, preparation, delivery, sourcing, portions, a food item, and/or the like) and/or user data (e.g., data associated with food data, including requesting a query, instructing the system 120 to tailor the system's output based on received criteria, and/or additional user dietary information, and/or the like) to the food data service 121 at (1). For example, a food data service 121 can receive user data and/or food data from food data storage server 130, user device(s) 102, and/or external system(s) 150. As an example, food data service 121 can receive user data from user device(s) 102 in response to a user request. Alternatively, or in addition, food data service 121 can receive user data and/or food data from food data storage server 130 based on one or more instructions generated by, for example, users using user device(s) 102. In an additional implementation, food data service 121 can receive user data and/or food data from external system(s) 150 based on a user input from external system(s) 150 and/or user device(s) 102. In an additional implementation, the food data storage server 130 may retrieve, from a data store (e.g., food data store 131), user data and/or food data including any and/or all of the data described herein.

In another implementation, the food data storage server 130 receives user data and/or food data from user device(s) 102. In another implementation, a user can transmit user data and/or food data from an interface of the user device(s) 102 to the external system(s) 150 and/or the food data storage server 130.

The food data service 121 may determine a recipe based on the transmitted food data and/or the user data at (2). Additionally and/or alternatively, the food data service 121 can determine one or more ingredients as part of a recipe based on the received user data and/or food data. For example, the food data service 121 can generate a query and search one or more databases for a list of ingredients for a specified recipe. Additionally, food data service 121 can determine a recipe based on text and/or an image received in response to, for example, a user selection on an interface of user device(s) 102. The image, text, and/or other data can include a menu, pricing, and an item description.

After the food data service 121 determines a recipe based on the transmitted food data and/or user data, the food data service 121 can retrieve nutrition data from nutrition data store 140 at (3). In various implementations, the nutrition data store 140 can be queried by the food data service 121 to generate a list of nutritional information for each ingredient of the determined recipe. In various implementations, the query generated by food data service 121 can include one or more aspects associated with the determined recipe, such as but not limited to preparation, delivery, sourcing, portion(s), a food item, and/or the like. In an alternative implementation, the food data service 121 can query one or more databases and/or another system such as, for example, food data storage server 130, food data store 131, user device(s) 102, and/or external system(s) 150. In an additional implementation, the food data service 121 can retrieve nutrition data from user data and/or food data. Advantageously, nutrition data can include a comprehensive list of nutrients for each ingredient of the determined recipe, customized for preparation, delivery, sourcing and/or the like of the recipe and the associated ingredients.

After the food data service 121 generates a comprehensive list of ingredients for a determined recipe along with associated nutrition data for each ingredient (e.g., nutrition data), the food data service 121 can transmit the nutrition data to the dietary processor 122.

After the dietary processor 122 receives the nutritional information, the dietary processor 122 can extract the nutritional information to perform various routines and/or dietary analysis at (5). As part of extracting the nutritional information, the dietary processor 122 can determine whether the information is complete and/or determine one or more instructions as part of the user data. For example, the dietary processor 122 can determine, based on the received user data, that the user requests that the nutritional information is compared with a target dietary motivation. In an additional implementation, the dietary processor 122 can determine, based on the user data, that the user is requesting that the data be generalized using the most common ingredients instead of analyzing the nutrition data for a specific recipe.

After the dietary processor 122 extracts the nutritional information, the dietary processor 122 can determine a fact summary for the determined recipe at (6). In various implementations, the dietary processor 122 can receive user data including, for example, a request from the user to generate a fact summary according to a user defined template. As an illustrative example, the dietary processor 122 can receive user data including a dietary motivation, and the dietary processor 122 can adjust a fact summary to include data associated with a dietary motivation (e.g., display of calories and/or sodium for a dietary motivation associated with a weight tracker, and/or a display protein and/or total carbohydrates for a dietary motivation associated with body building). Additionally, and or alternatively, the dietary processor 122 can generate a fact summary according to a system-generated template. In various implementations, the dietary processor 122 can generate a fact summary for two or more recipes. In various implementations, the dietary processor 122 can further compare the generated fact summaries for two or more recipes and transmit the comparison to, for example, user device(s) 102. In various implementations, a user may transmit a first recipe to the dietary processor 122, and then transmit one or more changes to the first recipe to the dietary processor 122. In various implementations, the dietary processor 122 can compare the first recipe to the second recipe in real-time and generate a fact summary displaying the nutritional changes from the first recipe to the second recipe.

The dietary processor 122 can further generate a nutricise at (7). The nutricise can be generated from, for example, the user data. Dietary processor 122 can additionally or alternatively generate a nutricise based on one or more dietary motivations.

After the dietary processor 122 has determined a nutricise, the dietary processor 122 can estimate a nutritional score at (8) in accordance with a nutritive model that may be specific to the user or generalized for a population of users. In various implementations, the dietary processor 122 can receive user data including, for example, a request from the user to generate a nutritional score according to a user defined dietary motivation. As an illustrative example, in response to receiving user data associated with a dietary motivation, the dietary processor 122 can, in the context of the nutritive model, adjust a nutritional score based on data (e.g., user data, food data, and/or nutrition data, and/or the like) associated with a dietary motivation (e.g., adjusting a score based on whether the received recipe meets a dietary need such as reduced sodium, or increased iron intake). Additionally, and or alternatively, the dietary processor 122 can generate a nutritional score according to a system-generated template. In various implementations, the dietary processor 122 can generate a nutritional score for two or more recipes. In various implementations, the dietary processor 122 can further compare the generated nutritional score for two or more recipes and transmit the comparison to, for example, user device(s) 102. In various implementations, a user may transmit a first recipe to the dietary processor 122, and then transmit one or more changes to the first recipe to the dietary processor 122. In various implementations, the dietary processor 122 can compare the first recipe to the second recipe in real-time and generate a nutritional score displaying changes to the nutritional score from the first recipe to the second recipe.

After the dietary processor 122 estimates a nutritional score for the received recipe based on the nutritional information, the dietary processor 122 can transmit the dietary data (e.g., the fact summary, nutricise, and/or nutrition score) and/or the nutritional information (e.g., the user data, the food data, and/or the nutrition data) to the nutrition optimizer 123 at (9).

After the nutrition optimizer 123 receives the dietary data and/or the nutritional information, the nutrition optimizer 123 can generate recommendation(s) based on the dietary data and/or nutritional information at (10). In various implementations, the nutrition optimizer 123 can generate an economic impact for one or more determined recipes. In various implementations, the nutrition optimizer 123 can determine an economic impact based on data associated with a recipe. For example, the nutrition optimizer 123 can extract and determine a cost associated with one or more nutrients, ingredients, preparation of the ingredients, sourcing of the ingredients and/or the like. Further, the nutrition optimizer 123 can generate an estimated cost associated with creating a new recipe and/or adjusting a recipe. The nutrition optimizer 123 can generate an estimated cost of a recipe based on, for example, user data, food data, and/or nutrition data. Additionally and/or alternatively, the nutrition optimizer 123 can utilize historical costs of a recipe, ingredients, preparation, sourcing, delivery, and/or the like, as part of user data, and/or food data to generate an estimated cost.

Additionally and/or alternatively, the nutrition optimizer 123 may generate one or more recommendation(s) based on the dietary data and/or the nutritional information. For example, the nutrition optimizer 123 can generate a recommendation to adjust a recipe to meet one or more user defined dietary motivations and/or to meet a user defined economic goal. As an illustrative example, nutrition optimizer 123 can receive dietary data (e.g., a recipe and instructions to recommend changes to a recipe based on a target cost savings). The nutrition optimizer 123 can, based on the received dietary data and/or nutrition data for the determined recipe, determine one or more recommendations to meet the user data's cost savings target.

After the nutrition optimizer 123 has generated recommendation(s) the nutrition optimizer 123 can further generate instruction(s) at (11). In various implementations, the nutrition optimizer 123 can generate instructions based on the received dietary data and/or the nutritional information. In various implementations, the nutrition optimizer 123 can generate instructions for external system(s) 150. As an illustrative example, nutrition optimizer 123 can generate instructions, based on for example, one or more recommendation(s), to add one or more ingredients to an acquisition list (e.g., a shopping cart). Further, in various implementations, the nutrition optimizer 123 can generate instruction(s) to update and/or revise a menu based on the one or more recommendation(s). Additionally and/or alternatively, nutrition optimizer 123 can generate instruction(s) for a restaurant management system to add one or more recipes, food items, and/or the like to a menu based on one or more determined recommendation(s).

Additionally, the nutrition optimizer 123 can generate instruction(s) and/or nudge a user as part of a diet management system. The nutrition optimizer 123 can, for example, receive dietary data and/or nutrition information from the dietary processor 122 including user data defining a user's dietary motivations. The nutrition optimizer 123 can determine for a selected recipe, one or more adjustments to the recipe to support the user's dietary motivations and/or determine whether the recipe meets the user's dietary motivations (e.g., supports healthy heart, supports a low sodium diet, and/or the like). The nutrition optimizer 123 can generate instructions for a user device to generate a nudge to the user, providing results of the analysis in accordance with the one or more user dietary motivations. Alternatively, the nutrition optimizer 123 may generate instruction(s) including one or more recommendation(s) such as, to display one or more alternative recipes that may meet the user's dietary motivations if the nutrition optimizer 123 determines that adjustment to the user's recipe may not meet the user dietary motivations.

Once the nutrition optimizer 123 generates recommendation(s) and/or instruction(s) (e.g., optimization data), the nutrition optimizer 123 can transmit the dietary data, nutritional information, and/or optimization data to food data storage server 130, user device(s) 102, and/or external system(s) 150 at (12). The optimization data and/or any other data can include for example, computer-executable code instructing the user device(s) 102 to display information according to the determined dietary data and/or optimization data (e.g., display a fact summary, a nutricise, and/or a nutritional score for one or more determined recipes, and/or display one or more recommendation(s) as described herein). In an example implementation, the nutrition optimizer 123 transmits instruction(s) including a nutritional score to the user device(s) 102 via network 110, where the user device(s) 102 may display the nutritional score and/or additional information associated with a determined recipe.

In various implementation, the nutrition optimizer 123 can transmit instructions to various external system(s) 150, including but not limited to an acquisition list (e.g., a shopping cart), a restaurant management system, and/or a diet management system. As an illustrative example, nutrition optimizer 123 can transmit instructions to add one or more ingredients, food items and/or the like, to a shopping cart. Further, in various implementations, the nutrition optimizer 123 can transmit instruction(s) to update and/or revise a menu based on the one or more recommendation(s). Additionally and/or alternatively, nutrition optimizer 123 can transmit instruction(s) for a restaurant management system to add one or more recipes, food items, and/or the like to a menu based on one or more determined recommendation(s). Additionally, the nutrition optimizer 123 can transmit instructions for a user device(s) 102 to generate a nudge to a as part of a diet management system. Alternatively and/or in addition, the nutrition optimizer 123 can transmit dietary data, nutritional information, and/or optimization data to a food data storage server 130. As an illustrative example, the nutrition optimizer 123 can transmit a history log of the determined recipe, one or more ingredients, a nutritional score for the determine recipe and/or any other data associated with the determined recipe (e.g., nutritional information, dietary data, and/or optimization data).

### Example Routine for Various Aspects of a Nutrition Analysis System

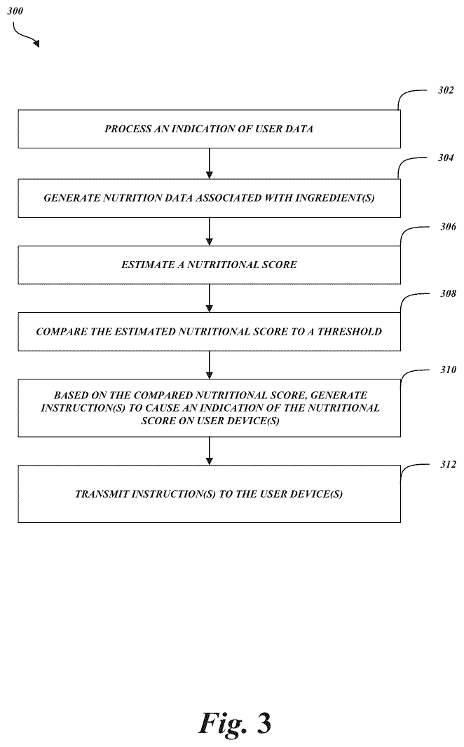

FIG. 3 is a flow diagram depicting an example nutritional score routine 300 illustratively implemented by a nutritional analysis system 120 according to various implementations. For example, the system 120 of FIG. 1 can be configured to execute routine 300. In various implementations, routine 300 can be executed after user device(s) 102 of FIG. 1 transmits user data to the food data service 121. The routine 300 begins at block 302.

At block 302, the system 120 can process an indication of user data. For example, food data service 121 can receive user data, including images, text and/or another form of data, including among other things, a meal, nutrients, ingredients, preparation, delivery, sourcing, portion(s), a food item, and/or the like. Additionally, user data can be received from, for example, user device(s) 102, external system(s) 150, and/or food data storage server 130. User data can include, for example, data associated with a food item, including requesting a query and/or instructing the system 120 to tailor the system's output based on user data. Example instructions associated with user data can include a request to determine a nutritional score based on an individual and/or based on the general population, a request to determine a general nutritional score and/or a custom score based on one or more dietary needs, such but not limited to, diabetes prevention, allergy limitations, muscle/body building, reduced sodium intake, calorie targets, portion control, increasing iron-rich foods, optimize vitamin D intake, enhance immune system function, increase energy levels, lower blood pressure, reduce risk of obesity, and/or the like. Further, user data can include instructions associated with a request to determine an economic impact and/or instructions to generate recommendations to change one or more ingredients based on for example, a target cost savings and/or one or more dietary needs associated with a food item. Additionally and/or alternatively, user data and/or food data as described herein can be interchangeably called and/or combined into “user data”. In various implementations, the system 120 can process an indication of user data including at least one recipe having one or more ingredients and a query, wherein the query is associated with at least one ingredient.

At block 304, the system 120 can generate nutrition data associated with the user data and/or food data of block 302. The nutrition data can be generated based on, for example, the results of a query as executed by food data service 121. In some implementations, nutrition data can be received from for example, user device(s) 102, external system(s) 150, and/or food data storage server 130. In some implementations, food data service 121 can query an internal and/or external database to obtain nutrition data associated with the received user data. For example, the food data service 121 can determine specifics on nutrients, ingredients, preparation, delivery, sourcing, and/or the like of one or more food items. The food data service 121 can query one or more nutrition databases, such as but not limited to an online database (e.g., USDA nutritional database, European Food Safety Authority Comprehensive European Food Consumption Database, Canadian Nutrient File, a private nutritional database, and/or the like), a restaurant management system, shopping carts, diet management systems, and/or any other system that generates nutrition data. As an illustrative example, nutrition data from one or more databases can include calories, carbohydrates, sugars, dietary fiber, protein, saturated fat, monounsaturated fat, polyunsaturated fat, cholesterol, sodium, potassium, calcium, iron, Vitamins (e.g., C, A, E), and/or the like. Further, nutrition data for each food item can vary based on one or more aspects of a food item, such as for example, ingredient, preparation, cooking time, servings yield, storage, reheating, and/or the like.

At block 306, the system 120 can estimate a nutritional score based on the user data, the nutrition data, and/or other data received by the system 120. In various implementations, dietary processor 122 can receive user data including, for example, a request from the user to estimate a nutritional score according to a user defined dietary motivation. As an illustrative example, in response to receiving user data associated with a dietary motivation, the dietary processor 122 can adjust a nutritional score of a food item based on data (e.g., user data, nutrition data, and/or the like) associated with a dietary motivation (e.g., adjusting a score based on whether the received recipe meets the dietary need such as a recipe to reduce sodium, or increase iron intake). Additionally, and or alternatively, the dietary processor 122 can estimate a nutritional score according to a system-generated template or model. In various implementations, the dietary processor 122 can estimate a nutritional score for two or more recipes.

At block 308, the system 120 can compare the estimated nutritional score to a threshold. The threshold can be, for example, determined by the system 120 and/or determined by user data, nutrition data, and/or another source. For example, a nutritional score can be compared to a threshold value, to determine whether the food item meets one or more dietary motivations. In various implementations, the dietary processor 122 can further compare the estimated nutritional score for two or more food items. In various implementations, the dietary processor 122 can compare a nutritional score for a first food item to a second nutritional score for a second food item in real-time. In various implementations, a user may transmit a first food item to the dietary processor 122, and then transmit one or more changes to the first food item to the dietary processor 122. In response to changes to the one or more food items, the dietary processor 122 can further determine and/or plot changes to the nutritional score.

At block 310, the system 120 generates instruction(s) to cause an indication of the nutritional score on the user device(s) 102. In some implementations, nutrition optimizer 123 can generate instruction(s) based on the results of a compared nutritional score to a dietary motivation, a threshold value, and/or another nutritional score. Additionally and/or alternatively, the nutrition optimizer 123 can generate, in response to the compared nutritional score to a threshold, instruction(s) to add one or more ingredients to a shopping cart. In various implementations, the nutrition optimizer 123 can generate, in response to the compared nutritional score(s), instruction(s) to revise a menu and/or add a food item to a menu of a restaurant management system. In various implementations, the nutrition optimizer 123 can generate, in response to the compared nutritional score(s), instructions to cause a nudge via a user device(s) 102, to indicate that the food item meets or does not meet at least one dietary motivation.

At block 312, the system 120 can transmit the generated instruction(s) to user device(s) 102. Additionally, and/or alternatively the system 120 (e.g., nutrition optimizer 123) can transmit the generated instruction(s) to external system(s) 150 and/or food data storage server 130. In some implementations, nutrition optimizer 123 can transmit instructions including the results of a comparison of two or more nutritional scores (e.g., compared to a dietary motivation, a threshold value, and/or another nutritional score) to user device(s) 102, external system(s) 150, and/or food data storage server 130, to cause the results to be displayed via a GUI. In various implementations, the nutrition optimizer 123 can transmit instruction(s) to cause external system(s) 150 to add one or more ingredients to a shopping cart. In various implementations, the nutrition optimizer 123 can transmit instruction(s) to cause external system(s) 150 to revise a menu, and/or to cause external system(s) 150 to add one or more food items to a menu. In various implementations, the nutrition optimizer 123 can transmit instruction(s) to cause external system(s) 150 to indicate that a determined food item meets or does not meet a dietary motivation.

### Additional Example Operational Environment of a Nutritional Analysis System

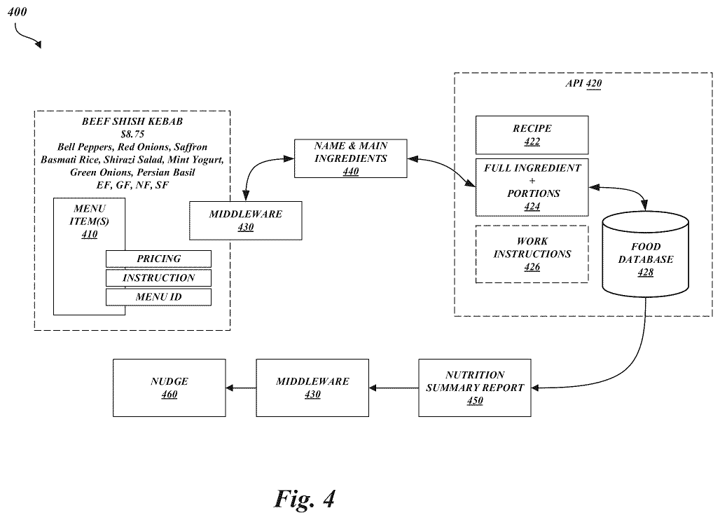

FIG. 4 is an additional example operational environment 400 for a nutritional analysis system 120. As illustrated in FIG. 4, an example operational environment 400 can include menu item(s) 410, middleware 430, name & main ingredients 440, an API 420, a nutrition summary report 450, and a nudge 460.

Menu items 410 can include, for example, pricing, instructions, and/or a menu ID for one or more menu items. The menu items 410 can be for example, similar to and/or the same as at least user data as described with reference to FIG. 1. Further, the menu items 410 can be compiled via, for example, middleware 430. Middleware can include be any software platform and/or communication protocol enabling communication between multiple platforms, such as between menu item(s) 410 and/or name & main ingredients 440, between nutrition summary report 450 and/or nudge 460, and/or between any other components of operational environment 400. In some examples middleware 430 is similar to network 110. Middleware 430 can receive menu items 410 and generate name & main ingredients 440. In some examples name & main ingredients 440 are similar to, and/or the same as at least user data and/or food data as described above with reference to FIG. 1. Once the middleware 430 generates name & main ingredients 440, the middleware 430 transfers the name & main ingredients 440 data to the API 420.

API 420 can be any application programming interface designed to accept name & main ingredient 440 data and determine for example, full ingredient & portions 424, a recipe 422, and/or work instructions 426. The API 420 can be the same as and/or similar to one or more aspects of at least the food data service 121, the dietary processor 122, the nutrition optimizer 123, and/or the food data storage server 130 as described with reference to FIG. 1. Further, the API can generate a recipe 422, full ingredient & portions 424, and/or work instructions 426, based on a query of a food database 428. Food database 428 can be the same as and/or similar to at least nutrition data store 140 and/or food data store 131 of FIG. 1. After the API 420 generates, based on a query of a food database 428, the recipe 422, the full ingredient & portions 424, and/or the work instructions 426, the API 420 can transmit the data to a nutrition summary report 450. The nutrition summary report 450 can generate a summary similar to and/or the same as at least dietary data, nutritional data, and/or optimization data generated by the food data service 121, the dietary processor 122 and/or nutrition optimizer 123 as described with reference to FIG. 1 and/or FIG. 2.

Further, after the nutrition summary report 450 is generated, the nutrition summary report 450 can be transmitted to middleware 430, where it can be further transmitted to nudge 460. At nudge 460 one or more actions may be executed in accordance with the nutrition summary report 450. In some examples, the actions at nudge 460 can be the same as and/or similar to transmitted instruction(s) of block 312 of FIG. 3., and/or any of the generated recommendation(s) and/or generated instruction(s) as illustrated or described at least by food data service 121, dietary processor 122, and/or nutrition optimizer 123 of FIG. 1.

### Additional Example Implementations and Details

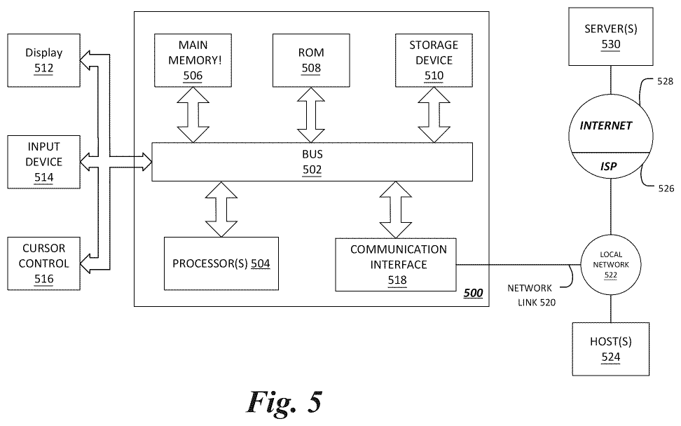

In an implementation the system (e.g., one or more aspects of the system 120, one or more aspects of the operating environment 100, and/or the like) may comprise, or be implemented in, a “virtual computing environment”. As used herein, the term “virtual computing environment” should be construed broadly to include, for example, computer-readable program instructions executed by one or more processors (e.g., as described in the example of FIG. 5) to implement one or more aspects of the modules and/or functionality described herein. Further, in this implementation, one or more services/modules/engines and/or the like of the system may be understood as comprising one or more rules engines of the virtual computing environment that, in response to inputs received by the virtual computing environment, execute rules and/or other program instructions to modify operation of the virtual computing environment. For example, a request received from a user computing device may be understood as modifying operation of the virtual computing environment to cause the request access to a resource from the system. Such functionality may comprise a modification of the operation of the virtual computing environment in response to inputs and according to various rules. Other functionality implemented by the virtual computing environment (as described throughout this disclosure) may further comprise modifications of the operation of the virtual computing environment, for example, the operation of the virtual computing environment may change depending on the information gathered by the system. Initial operation of the virtual computing environment may be understood as an establishment of the virtual computing environment. In some implementations the virtual computing environment may comprise one or more virtual machines, containers, and/or other types of emulations of computing systems or environments. In some implementations the virtual computing environment may comprise a hosted computing environment that includes a collection of physical computing resources that may be remotely accessible and may be rapidly provisioned as needed (commonly referred to as “cloud” computing environment).

Implementing one or more aspects of the system as a virtual computing environment may advantageously enable executing different aspects or modules of the system on different computing devices or processors, which may increase the scalability of the system. Implementing one or more aspects of the system as a virtual computing environment may further advantageously enable sandboxing various aspects, data, or services/modules of the system from one another, which may increase security of the system by preventing, e.g., malicious intrusion into the system from spreading. Implementing one or more aspects of the system as a virtual computing environment may further advantageously enable parallel execution of various aspects or modules of the system, which may increase the scalability of the system. Implementing one or more aspects of the system as a virtual computing environment may further advantageously enable rapid provisioning (or de-provisioning) of computing resources to the system, which may increase scalability of the system by, e.g., expanding computing resources available to the system or duplicating operation of the system on multiple computing resources. For example, the system may be used by thousands, hundreds of thousands, or even millions of users simultaneously, and many megabytes, gigabytes, or terabytes (or more) of data may be transferred or processed by the system, and scalability of the system may enable such operation in an efficient and/or uninterrupted manner.

Various implementations of the present disclosure may be a system, a method, and/or a computer program product at any possible technical detail level of integration. The computer program product may include a computer-readable storage medium (or mediums) having computer-readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer-readable storage medium (or mediums). Computer-readable storage mediums may also be referred to herein as computer-readable storage or computer-readable storage devices.

The computer-readable storage medium can be a tangible device that can retain and store data and/or instructions for use by an instruction execution device. The computer-readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions (as also referred to herein as, for example, “code,” “instructions,” “module,” “application,” “software application,” “service,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, and/or the like, and procedural programming languages, such as the “C” programming language or similar programming languages. Computer-readable program instructions may be callable from other instructions or from itself, and/or may be invoked in response to detected events or interrupts. Computer-readable program instructions configured for execution on computing devices may be provided on a computer-readable storage medium, and/or as a digital download (and may be originally stored in a compressed or installable format that requires installation, decompression, or decryption prior to execution) that may then be stored on a computer-readable storage medium. Such computer-readable program instructions may be stored, partially or fully, on a memory device (e.g., a computer-readable storage medium) of the executing computing device, for execution by the computing device. The computer-readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some implementations, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to implementations of the disclosure. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart(s) and/or block diagram(s) block or blocks.

The computer-readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowcharts and block diagrams in the Figures illustrate the architecture, functionality, and operation of possible implementations of systems, methods, and computer program products according to various implementations of the present disclosure. In this regard, each block in the flowchart or block diagrams may represent a service, module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. In addition, certain blocks may be omitted or optional in some implementations. The methods and processes described herein are also not limited to any particular sequence, and the blocks or states relating thereto can be performed in other sequences that are appropriate.

It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. For example, any of the processes, methods, algorithms, elements, blocks, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, and/or the like with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above implementations may generally (but not necessarily) be controlled and/or coordinated by operating system software, such as Mac OS, IOS, Android, Chrome OS, Windows OS (e.g., Windows XP, Windows Vista, Windows 7, Windows 8, Windows 10, Windows 11, Windows Server, and/or the like), Windows CE, Unix, Linux, SunOS, Solaris, Blackberry OS, VxWorks, or other suitable operating systems. In other implementations, the computing devices may be controlled by a proprietary operating system. Conventional operating systems control and schedule computer processes for execution, perform memory management, provide file system, networking, I/O services, and provide a user interface functionality, such as a GUI, among other things.

For example, FIG. 5 shows a block diagram that illustrates a computer system 500 upon which various implementations and/or aspects (e.g., one or more aspects of the operating environment 100, one or more aspects of the system 120, one or more aspects of the user device(s) 102, one or more aspects of the external system(s) 150, one or more aspects of the food data storage server 130, and/or the like) may be implemented. Multiple such computer systems 500 may be used in various implementations of the present disclosure. Computer system 500 includes a bus 502 or other communication mechanism for communicating information, and a hardware processor, or multiple processors, 504 coupled with bus 502 for processing information. Hardware processor(s) 504 may be, for example, one or more general purpose microprocessors.

Computer system 500 also includes a main memory 506, such as a random-access memory (RAM), cache and/or other dynamic storage devices, coupled to bus 502 for storing information and instructions to be executed by processor 504. Main memory 506 also may be used for storing temporary variables or other intermediate information during execution of instructions to be executed by processor 504. Such instructions, when stored in storage media accessible to processor 504, render computer system 500 into a special-purpose machine that is customized to perform the operations specified in the instructions. The main memory 506 may, for example, include instructions to implement server instances, queuing modules, memory queues, storage queues, user interfaces, and/or other aspects of functionality of the present disclosure, according to various implementations.

Computer system 500 further includes a read only memory (ROM) 508 or other static storage device coupled to bus 502 for storing static information and instructions for processor 504. A storage device 510, such as a magnetic disk, optical disk, or USB thumb drive (Flash drive), and/or the like, is provided and coupled to bus 502 for storing information and instructions.

Computer system 500 may be coupled via bus 502 to a display 512, such as a cathode ray tube (CRT) or LCD display (or touch screen), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane. In some implementations, the same direction information and command selections as cursor control may be implemented via receiving touches on a touch screen without a cursor.

Computing system 500 may include a user interface module to implement a GUI that may be stored in a mass storage device as computer executable program instructions that are executed by the computing device(s). Computer system 500 may further, as described below, implement the techniques described herein using customized hard-wired logic, one or more ASICs or FPGAs, firmware and/or program logic which in combination with the computer system causes or programs computer system 500 to be a special-purpose machine. According to one implementation, the techniques herein are performed by computer system 500 in response to processor(s) 504 executing one or more sequences of one or more computer-readable program instructions contained in main memory 506. Such instructions may be read into main memory 506 from another storage medium, such as storage device 510. Execution of the sequences of instructions contained in main memory 506 causes processor(s) 504 to perform the process steps described herein. In alternative implementations, hard-wired circuitry may be used in place of or in combination with software instructions.

Various forms of computer-readable storage media may be involved in carrying one or more sequences of one or more computer-readable program instructions to processor 504 for execution. For example, the instructions may initially be carried on a magnetic disk or solid-state drive of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN (or WAN component to communicated with a WAN). Wireless links may also be implemented. In any such implementation, communication interface 518 sends and receives electrical, electromagnetic, or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the worldwide packet data communication network now commonly referred to as the “Internet” 528. Local network 522 and Internet 528 both use electrical, electromagnetic, or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518.

The received code may be executed by processor 504 as it is received, and/or stored in storage device 510, or other non-volatile storage for later execution.

As described above, in various implementations certain functionality may be accessible by a user through a web-based viewer (such as a web browser), or other suitable software program). In such implementations, the user interface may be generated by a server computing system and transmitted to a web browser of the user (e.g., running on the user's computing system). Alternatively, data (e.g., user interface data) necessary for generating the user interface may be provided by the server computing system to the browser, where the user interface may be generated (e.g., the user interface data may be executed by a browser accessing a web service and may be configured to render the user interfaces based on the user interface data). The user may then interact with the user interface through the web-browser. User interfaces of certain implementations may be accessible through one or more dedicated software applications. In certain implementations, one or more of the computing devices and/or systems of the disclosure may include mobile computing devices, and user interfaces may be accessible through such mobile computing devices (for example, smartphones and/or tablets).

Systems discussed herein may include a disease management system. Example disease management systems are discussed in U.S. Publication No. 2023/0115397, which is incorporated herein by reference in its entirety.

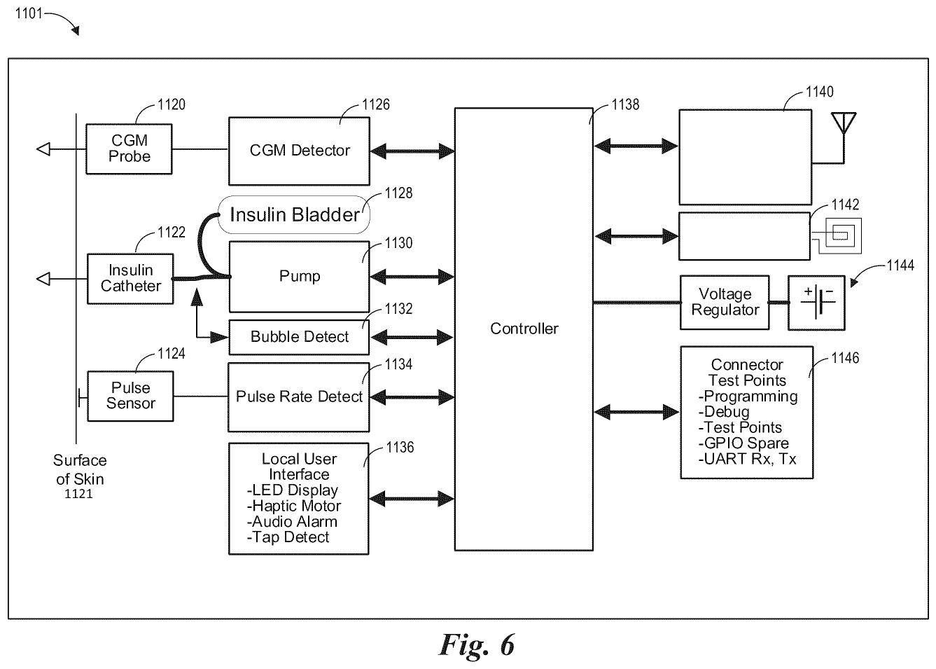

FIG. 6 shows a block diagram of an example disease management system 1101. In some examples, the disease management system 1101 may be part of a disease management environment, such as described above. A disease management system 1101 may be configured to measure one or more physiological parameters of a patient (such as pulse, skin temperature, or other values), measure one or more analytes present in the blood of a patient (such as glucose, lipids, or other analyte) and administer medication (such as insulin, glucagon, or other medication). In some examples, a disease management system 1101 may be configured to communicate with one or more hardware processors that may be external to the disease management system 1101, such as a cloud-based processor or user device. A disease management system 1101 may include an NFC tag to support authentication and pairing with a user device (for example, smart phone or smart watch), Bluetooth communication with additional disease management systems or devices, and Bluetooth communication with a paired user device running an associated control application. To support ease of use and safe interaction with the patient, the system may incorporate user input through a tap-detecting accelerometer and provide feedback via an audio speaker, haptic vibration, and/or optical indicators. The system may operate on battery power and support both shelf-life and reliable operation once applied to the patient. Battery life may be managed through control of several planned levels of sleep and power consumption. To support this reliability, a controller can monitor several system-health parameters, and monitor temperatures of the included medication, and ambient temperature for the life of the device.

As illustrated in FIG. 6, a controller 1138 of the disease management system 1101 may be configured to communicate and control one or more components of the disease management system 1101. The controller 1138 may include one or more hardware processors, such as a printed circuit board (PCB) or the like. The controller 1138 may be configured to communicate with peripheral devices or components to support the accurate measurement of physiological parameters and blood analytes, such as patient pulse, temperature, and blood glucose, using detector electronics. The controller 1138 may subsequently calculate dose or receive a calculated dose value and administer medication, such as insulin, by actuation of an actuated pump. The controller 1138 may record device activity and transfer the recorded data to non-volatile secure memory space. At the end of the life of a device or system, the controller can be configured to lock operation, and create a data recovery module to permit authenticated access to the recorded data if needed.

A disease management system 1101 may include an analyte sensor 1120. The analyte sensor 1120 may be configured to detect analytes in the patient's blood. For example, an analyte sensor 1120 can include a glucose sensing probe configured to pierce the surface of the skin 1121. In some examples, a disease management system 1101 may include a plurality of analyte sensors 1120 to detect one or more analytes. In some examples, an analyte sensor 1120 may be configured to detect a plurality of analytes. Sensed analytes may include, but are not limited to, glucose, insulin, and other analytes. An analyte sensor 1120 may be configured to communicate with an analyte detector 1126. The analyte detector 1126 may be configured to receive a signal of one or more analyte sensors 1120 in order to measure one or more analytes in the blood of the patient. The analyte detector 1126 may be configured to communicate with the controller 1138. For example, the analyte detector 1126 may be configured to, for example, send analyte values to the controller 1138 and receive control signals from the controller.

A disease management system 1101 may include a medication catheter 1122. The medication catheter 1122 may be configured to administer medication, including, but not limited to insulin, to the patient. The medication catheter 1122 may receive medication from a medication bladder 1128 configured to contain medication to be administered. The medication bladder 1128 may be configured to contain medication for a prolonged period, such as 1 day, 3 days, 6 days, or more. The medication bladder 1128 may be configured to contain certain medication types, such as insulin. In some examples, a disease management system 1101 may include a plurality of medication bladders 1128 for one or more reservoirs of the same or different medications. In some examples, a disease management system 1101 may be configured to mix medications from medication bladders 1128 prior to administration to the patient. A pump 1130 may be configured to cause medication to be administered from the bladder 1128 to the patient through the insulin catheter 1122. A pump 1130 may include, but is not limited to, a pump such as described herein. The disease management system 1101 may include a bubble detection sensor 1132 that can detect bubbles in the liquid drug within any or multiple of fluid lines connecting the medication bladder 1128, pump 1130, and medication catheter 1122.

A disease management system 1101 may optionally include a physiological sensor 1124. The physiological sensor 1124 may include a pulse rate sensor, temperature sensor, pulse oximeter, the like or a combination thereof. In some examples, a disease management system 1101 may be configured to include a plurality of physiological sensors. The physiological sensor 1124 may be configured to communicate with a physiological detector 1134. The physiological detector 1134 may be configured to receive a signals of the physiological sensor 1124. The physiological detector 1134 may be configured to measure or determine and communicate a physiological value from the signal. The physiological detector 1134 may be configured to communicate with the controller 1138. For example, the physiological detector 1134 may be configured to, for example, send measured physiological values to the controller 1138 and receive control signals from the controller.

A disease management system 1101 may include one or more local user interfacing components 1136. For example, a local user interfacing component 1136 may include, but is not limited to one or more optical displays, haptic motors, audio speakers, and user input detectors. In some examples, an optical display may include an LED light configured to display a plurality of colors. In some examples, an optical display may include a digital display of information associated with the disease management system 1101, including, but not limited to, device status, medication status, patient status, measured analyte or physiological values, the like or a combination thereof. In some examples, a user input detector may include an inertial measurement unit, tap detector, touch display, or other component configured to accept and receive user input. In some examples, audio speakers may be configured to communicate audible alarms related to device status, medication status user status, the like or a combination thereof. A controller 1138 may be configured to communicate with the one or more local interfacing components 1136 by, for example, receiving user input from the one or more user input components or sending control signals to, for example, activate a haptic motor, generate an output to the optical display, generate an audible output, or otherwise control one or more of the local user interfacing components 1136.

A disease management system 1101 may include one or more communication components 1140. A communication component 1140 can include but is not limited to one or more radios configured to emit Bluetooth, cellular, Wi-Fi, or other wireless signals. In some examples, a communication component 1140 can include a port for a wired connection. Additionally, a disease management system 1101 may include an NFC tag 1142 to facilitate in communicating with one or more hardware processors. The one or more communication components 1140 and NFC tag 1142 may be configured to communicate with the controller 1138 in order to send and/or receive information associated with the disease management system 1101. For example, a controller 1138 may communicate medication information and measured values through the one or more communication components 1140 to an external device. Additionally, the controller 1138 may receive instructions associated with measurement sampling rates, medication delivery, or other information associated with operation of the management system 1101 through the one or more communication components 1140 from one or more external devices.

A disease management system 1101 may include one or more power components 1144. The power components may include but are not limited to one or more batteries and power management components, such as a voltage regulator. Power from the one or more power components 1144 may be accessed by the controller and/or other components of the disease management system 1101 to operate the disease management system 1101.

A disease management system 1101 may have one or more power and sleep modes to help regulate power usage. For example, a disease management system 1101 may have a sleep mode. The sleep mode may be a very low power mode with minimal functions, such as the RTC (or real time clock) and alarms to wake the system and take a temperature measurement of the system, or the like. In another example, a disease management system 1101 may include a measure temperature mode which may correspond to a low power mode with reduced functions. The measure temperature mode may be triggered by the RTC where the system is configured to take a temperature measurement, save the value, and return the system to a sleep mode. In another example, a disease management system 1101 may include a wake-up mode. The wake-up mode may be triggered by an NFC device and allow the system to pair with an external device with, for example, Bluetooth. If a pairing event does not occur, the system may return to sleep mode. In another example, a disease management system 1101 may include a pairing mode. The pairing mode may be triggered by an NFC device. When a controlling application is recognized, the system may proceed to pair with the application and set the system to an on condition and communicate to the cloud or other external device to establish initial data movement. In another example, a disease management system 1101 may include a rest mode where the system is configured to enter a lower power mode between measurements. In another example, a disease management system 1101 may include a data acquisition mode where the system is configured to enter a medium power mode where data acquisition takes place. In another example, a disease management system 1101 may include a parameter calculation mode where the system is configured to enter a medium power mode where parameter calculations, such as a blood glucose calculation, are performed and data is communicated to an external device and/or the cloud. In another example, a disease management system 1101 may include a pump mode where the system is configured to enter a higher power mode where the pump draws power to deliver medication to the patient.

A disease management system 1101 may include one or more connector test points 1146. The connecter test points may be configured to aid in programming, debugging, testing or other accessing of the disease management system 1101. In some examples, connector test points 1146 may include, for example, a GPIO spare, UART receiver or transmitter, the like or a combination thereof.

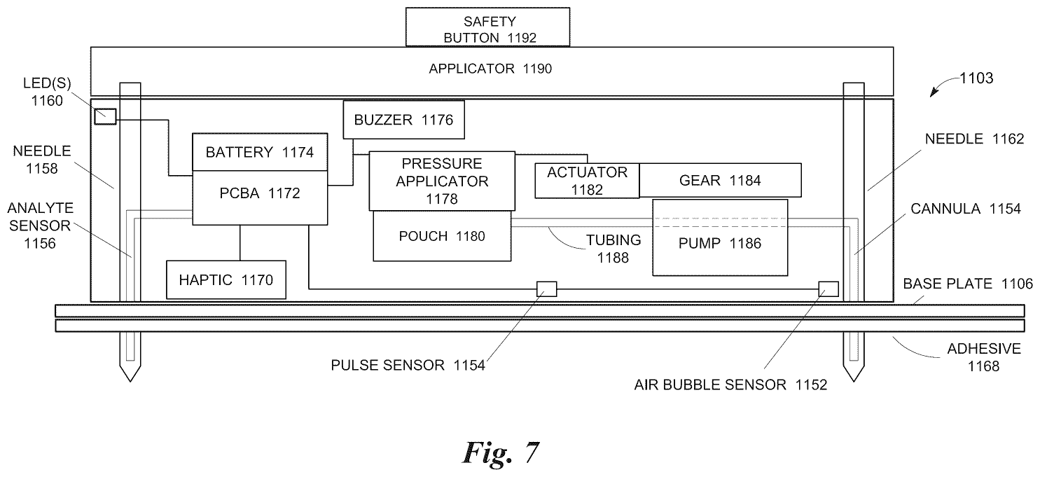

FIG. 7 illustrates an example implementation of a disease management system 1103 and applicator 1190 for applying a disease management system 1103 to a patient. Disease management system 1103 can include any one or more of the features discussed above with respect to the disease management system 1101 in addition to the features described below. In the illustrated example, an applicator 1190 may be configured to mate with the disease management system 1103. In some examples, an applicator 1190 may include a safety button 1192 for release or other interaction with the applicator 1190. In the illustrated example, a disease management system 1103 may include one or more LEDs 1160 that may be configured to output information using one or more of color, frequency, and length of display. In some examples, the disease management system 1103 may include a buzzer 1176, haptic actuator 1170, or other feedback mechanism, such as a speaker to output information to the patient, such as an alarm. In some examples, a disease management system 1103 may include a battery 1174, controller 1172. In some examples, a disease management system 1103 may include aspects of a medication administration system, such as a bladder 1180, a bladder pressure applicator 1178 to provide pressure on the bladder (such as a component of a pump), actuator 1182, pump gears 1184, and a pump 1186. In some examples, a disease management system 1103 may include one or more needles 1158 that may include one or more analyte sensors (such as a glucose sensor) 1156. In some examples, a disease management system 1103 may include one or more needles 1162 that may include one or more cannulas 1164 configured to administer medication to the patient. In some examples, a disease management system 1103 may include an air bubble sensor 1152 configured to detect the presence of air bubbles in the medication prior to delivery to the patient. In some examples, a glucose control system 1103 may include one or more physiological sensors 1154, such as a non-invasive physiological sensor including but not limited to a pulse sensor. In some examples, the disease management system 1103 may include a base plate 1106 and an adhesive layer 1168 below the base plate 1106 to provide adhesion of the disease management system 1103 to the patient's skin. As described below, a housing of the disease management system 1103 may consist of a combination of flexible and rigid material so as to both provide support for the components of the disease management system 1103 and allow conforming, at least in part, of the disease management system 1103 to the skin of the patient.

The adhesive layer 1168 may be configured to provide adhesion for a prolonged period. For example, the adhesive layer 1168 may be configured to adhere the disease management system 1103 to the skin of a patient for a period of 1 day, 3 days, 6 days, or more or fewer days or hours. In some examples, the adhesive layer may be configured to have an adhesive force sufficient to prevent accidental removal or movement of the disease management system 1103 during the intended period of use of the disease management system 1103. In some examples, the adhesive layer 1168 may be a single layer of adhesive across at least a portion of a surface the disease management system 1103 that is configured to interface with the patient. In some examples, the adhesive layer 1168 may include a plurality of adhesive areas on a surface of the disease management system 1103 that is configured to interface with the patient. In some examples, the adhesive layer 1168 may be configured to be breathable, adhere to the patient's skin after wetting by humidity or liquids such as tap water, saltwater, and chlorinated water. A thickness of the adhesive may be, for example, in a range of 0.1 to 0.5 mm or in a range of more or less thickness.

In some examples, a needle 1158, 1162 may be inserted at different depths based on a patient age, weight, or other parameter. For example, a depth of insertion of a medication cannula may be approximately 3 mm for 7 to 12 year olds. In another example, a depth of insertion of a medication cannula may be approximately 4 mm for 13 year olds and older. In another example, a depth of insertion of a medication needle may be approximately 4 to 4.5 mm for 7 to 12 year olds. In another example, a depth of insertion of a medication needle may be approximately 5 to 5.5 mm for 13 year olds and older. In another example, a depth of insertion of an analyte sensor may be approximately 3 mm for 7 to 12 year olds. In another example, a depth of insertion of an analyte sensor may be approximately 4 mm for 13year olds and older. In another example, a depth of insertion for a needle associated with an analyte sensor may be approximately 4 to 4.5 mm for 7 to 12 year olds. In another example, a depth of insertion for a needle associated with an analyte sensor may be approximately 5 to 5.5 mm for 13 year olds and older. However, other values or ranges for any of the inserted components are also possible.

Many variations and modifications may be made to the above-described implementations, the elements of which are to be understood as being among other acceptable examples. All such modifications and variations are intended to be included herein within the scope of this disclosure. The foregoing description details certain implementations. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Conditional language, such as, among others, “can,” “could,” “might,” or “may,” unless specifically stated otherwise, or otherwise understood within the context as used, is generally intended to convey that certain implementations include, while other implementations do not include, certain features, elements, and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation.

The term “substantially” when used in conjunction with the term “real-time” forms a phrase that will be readily understood by a person of ordinary skill in the art. For example, it is readily understood that such language will include speeds in which no or little delay or waiting is discernible, or where such delay is sufficiently short so as not to be disruptive, irritating, or otherwise vexing to a user.

Conjunctive language such as the phrase “at least one of X, Y, and Z,” or “at least one of X, Y, or Z,” unless specifically stated otherwise, is to be understood with the context as used in general to convey that an item, term, and/or the like may be either X, Y, or Z, or a combination thereof. For example, the term “or” is used in its inclusive sense (and not in its exclusive sense) so that when used, for example, to connect a list of elements, the term “or” means one, some, or all of the elements in the list. Thus, such conjunctive language is not generally intended to imply that certain implementations require at least one of X, at least one of Y, and at least one of Z to each be present.

The term “a” as used herein should be given an inclusive rather than exclusive interpretation. For example, unless specifically noted, the term “a” should not be understood to mean “exactly one” or “one and only one”; instead, the term “a” means “one or more” or “at least one,” whether used in the claims or elsewhere in the specification and regardless of uses of quantifiers such as “at least one,” “one or more,” or “a plurality” elsewhere in the claims or specification.

The term “comprising” as used herein should be given an inclusive rather than exclusive interpretation. For example, a general-purpose computer comprising one or more processors should not be interpreted as excluding other computer components, and may possibly include such components as memory, input/output devices, and/or network interfaces, among others.

While the above detailed description has shown, described, and pointed out novel features as applied to various implementations, it may be understood that various omissions, substitutions, and changes in the form and details of the devices or processes illustrated may be made without departing from the spirit of the disclosure. As may be recognized, certain implementations of the inventions described herein may be embodied within a form that does not provide all of the features and benefits set forth herein, as some features may be used or practiced separately from others. The scope of certain inventions disclosed herein is indicated by the appended claims rather than by the foregoing description. All changes which come within the meaning and range of equivalency of the claims are to be embraced within their scope.

### Example Clauses

Examples of the implementations of the present disclosure can be described in view of the following example clauses. The features recited in the below example implementations can be combined with additional features disclosed herein. Furthermore, additional inventive combinations of features are disclosed herein, which are not specifically recited in the below example implementations, and which do not include the same features as the specific implementations below. For sake of brevity, the below example implementations do not identify every inventive aspect of this disclosure. The below example implementations are not intended to identify key features or essential features of any subject matter described herein. Any of the example clauses below, or any features of the example clauses, can be combined with any one or more other example clauses, or features of the example clauses or other features of the present disclosure.
Clause 1. A system for estimating a nutritional score for a food item, the system comprising: memory that stores computer-executable instructions; and a processor in communication with the memory, wherein the computer-executable instructions, when executed by the processor, cause the processor to: process an indication of user data, wherein the user data comprises a food item including at least one recipe having one or more ingredients and a query, wherein the query is associated with at least one ingredient; generate, based at least in part on the query, nutrition data associated with the at least one ingredient, wherein the nutrition data comprises preparation data, sourcing data, delivery data, and a list of nutrients for the at least one ingredient; estimate, based at least in part on the nutrition data associated with the least one ingredient, a nutritional score; compare the estimated nutritional score to a threshold; based on the compared nutritional score, generate one or more instructions to cause an indication of the nutritional score on a user device; and transmit the generated one or more instructions to the user device.
Clause 2. The system of Clause 1, wherein the user data further comprises a dietary motivation data, and wherein the computer-executable instructions, when executed, further cause the processor to: estimate, based at least in part on the nutrition data associated with the at least one ingredient and the dietary motivation data, the nutritional score; based on the dietary motivation data, determine a threshold; compare the estimated nutritional score to a threshold; generate one or more instructions based on the nutritional score, or the dietary motivation data; and transmit the generated one or more instructions to cause an indication of the nutritional score on a user device.
Clause 3. The system of Clause 1, wherein the computer-executable instructions, when executed, further cause the processor to: based on the compared nutritional score, generate an instruction to add one or more ingredients to a shopping cart; and transmit the generated instruction to add one or more ingredients to the shopping cart, to cause an external system to purchase at least one of the one or more ingredients.
Clause 4. The system of Clause 1, wherein the computer-executable instructions, when executed, further cause the processor to: based on the compared nutritional score, generate an instruction to revise a menu; and transmit the generated instruction to cause an external system to revise a menu.
Clause 5. The system of Clause 1, wherein the computer-executable instructions, when executed, further cause the processor to: based on the compared nutritional score, generate an instruction to add one or more food items to a menu; and transmit the generated instruction, to cause an external system to add one or more food items to a menu.
Clause 6. The system of Clause 1, wherein the computer-executable instructions, when executed, further cause the processor to: based on the compared nutritional score, generate a nudge via a user device, indicating that the food item meets or does not meet at least one dietary motivation; and transmit the generated nudge, to cause a user device to indicate that a determined food item meets or does not meet a dietary motivation.
Clause 7. The system of Clauses 1-6, wherein the computer-executable instructions, when executed, further cause the processor to: process an indication of a second food item, wherein the second food item includes at least one recipe having one or more ingredients; generate, based at least in part on the query, a second nutrition data associated with the at least one ingredient of the second food item, wherein the nutrition data comprises preparation data, sourcing data, delivery data, and a list of nutrients for the at least one ingredient of the second food item; estimate, based at least in part of the nutrition data associated with the at least one ingredient of the second food item, a second nutrition score; compare the nutrition score and the second nutrition score; and based on the compared nutrition score and the second nutrition score, transmit one or more instructions to cause an indication of the compared nutritional scores on a user device.
Clause 8. The system of Clause 7, wherein the user data further comprises a dietary motivation, and wherein the computer-executable instructions, when executed, further cause the processor to: estimate, based at least in part on the second nutrition data and the dietary motivation data, a second nutritional score; based on the dietary motivation data, determine a threshold; compare the estimated second nutritional score to a threshold; generate one or more instructions based on the second nutritional score, or the dietary motivation data; and transmit the generated one or more instructions to further cause an indication of the second nutritional score on a user device.
Clause 9. The system of Clauses 8, wherein the motivation data further comprises an instruction to generate the nutritional score according to a user or a general population.
Clause 10. The system of Clauses 8, wherein the dietary motivation data comprises a threshold wherein the threshold is based at least in part on a nutritional analysis for diabetes prevention, allergy limitations, muscle/body building, reduced sodium intake, calorie targets, portion control, increasing iron-rich foods, optimize vitamin D intake, enhance immune system function, increase energy levels, lower blood pressure, or reduce risk of obesity.
Clause 11. The system of Clauses 1-8, wherein the list of nutrients comprises calories, carbohydrates, sugars, dietary fiber, protein, saturated fat, monounsaturated fat, polyunsaturated fat, cholesterol, sodium, potassium, calcium, iron, or Vitamins.
Clause 12. The system of Clauses 1-11, wherein the computer-executable instructions, when executed, further cause the processor to: estimate a fact summary based at least in part on the nutrition data associated with the at least one ingredient, wherein the fact summary comprises a name of a recipe, a description of the preparation, delivery, and sourcing of the at least one ingredient, and a list of nutrients associated with the at least one ingredient.
Clause 13. The system of Clauses 1-11, wherein the computer-executable instructions, when executed, further cause the processor to: estimate, based at least in part on the nutrition data associated with the at least one ingredient, a nutricise, wherein the nutricise comprises a learning activity that can help users understand healthy eating habits that incorporates physical fitness into the activity.
Clause 14. The system of Clauses 1-13, wherein the computer-executable instructions, when executed, further cause the processor to estimate an economic impact based on the nutrition data.
Clause 15. A system comprising: one or more computer-readable storage mediums having program instructions embodied therewith; and one or more processors configured to execute the program instructions to cause the system to perform the computer-implemented method of any of Clauses 1-14.
Clause 16. A computer program product comprising one or more computer-readable storage mediums having program instructions embodied therewith, the program instructions executable by one or more processors to cause the one or more processors to perform the computer-implemented method of any of Clauses 1-14.
Clause 17. A system as described herein.

What is claimed is:

1. A system for determining a health benefit to a user of a food item by estimating a nutritive score of the food item, the system comprising:
memory that stores computer-executable instructions; and
a processor in communication with the memory, wherein the computer-executable instructions, when executed by the processor, cause the processor to:
process an indication of user data, wherein the user data comprises a food item including at least one recipe having one or more ingredients and a query of a nutritive database, wherein the query is associated with at least one ingredient of the recipe;
generate, based at least in part on the query, nutritive data associated with the at least one ingredient, wherein the nutritive data comprises one or more of preparation data, sourcing data, delivery data, and a nutritive composition for the at least one ingredient;
initialize a nutritive model;
estimate, based at least in part on the nutritive data associated with the least one ingredient and the nutritive model, a nutritive score;
compare the estimated nutritive score to a threshold;
based on the compared nutritive score, generate one or more instructions to cause display of the nutritive score on a user device; and
transmit the generated one or more instructions to the user device.

2. The system of claim 1, wherein the user data further comprises a diet need data, and wherein the computer-executable instructions, when executed, further cause the processor to:
estimate, based at least in part on the nutritive data associated with the at least one ingredient, the diet need data, and the nutritive model, the nutritive score;
based on the diet need data, determine a threshold;
compare the estimated nutritive score to a threshold;
generate one or more instructions based on the nutritive score, or the diet need data; and
transmit the generated one or more instructions to cause an indication of the nutritive score on a user device.

3. The system of claim 1, wherein the computer-executable instructions, when executed, further cause the processor to:
based on the compared nutritive score, generate an instruction to add one or more ingredients to an acquisition list; and
transmit the generated instruction to add one or more ingredients to the acquisition list, to cause an external system to purchase at least one of the one or more ingredients.

4. The system of claim 1, wherein the computer-executable instructions, when executed, further cause the processor to:
based on the compared nutritive score, generate an instruction to revise a menu; and
transmit the generated instruction to cause an external system to revise a menu.

5. The system of claim 1, wherein the computer-executable instructions, when executed, further cause the processor to:
based on the compared nutritive score, generate an instruction to add one or more food items to a menu; and
transmit the generated instruction, to cause an external system to add one or more food items to a menu.

6. The system of claim 1, wherein the computer-executable instructions, when executed, further cause the processor to:
based on the compared nutritive score, generate a nudge via a user device, indicating that the food item meets or does not meet at least one dietary need; and
transmit the generated nudge, to cause a user device to indicate that a determined food item meets or does not meet a dietary need.

7. The system of claim 1, wherein the computer-executable instructions, when executed, further cause the processor to:
process an indication of a second food item, wherein the second food item includes at least one recipe having one or more ingredients;
generate, based at least in part on the query, a second nutrition data associated with the at least one ingredient of the second food item, wherein the nutrition data comprises preparation data, sourcing data, delivery data, and a list of nutrients for the at least one ingredient of the second food item;
estimate, based at least in part of the nutrition data associated with the at least one ingredient of the second food item, a second nutritive score;
compare the nutritive score and the second nutritive score; and
based on the compared nutritive score and the second nutritive score, transmit one or more instructions to cause an indication of the compared nutritive scores on a user device.

8. The system of claim 7, wherein the user data further comprises a dietary need data, and wherein the computer-executable instructions, when executed, further cause the processor to:
estimate, based at least in part on the second nutrition data and the diet need data, a second nutritive score;
based on the diet need data, determine a threshold;
compare the estimated second nutritive score to a threshold;
generate one or more instructions based on the second nutritive score, or the diet need data; and
transmit the generated one or more instructions to further cause an indication of the second nutritive score on a user device.

9. The system of claim 8, wherein the diet need data further comprises an instruction to generate the nutritive score according to a user or a general population.

10. The system of claim 8, wherein the diet need data comprises a threshold wherein the threshold is based at least in part on a nutritional analysis for diabetes prevention, allergy limitations, muscle/body building, reduced sodium intake, calorie targets, portion control, increasing iron-rich foods, optimize vitamin D intake, enhance immune system function, increase energy levels, lower blood pressure, or reduce risk of obesity.

11. The system of claim 1, wherein the list of nutrients comprises calories, carbohydrates, sugars, dietary fiber, protein, saturated fat, monounsaturated fat, polyunsaturated fat, cholesterol, sodium, potassium, calcium, iron, or Vitamins.

12. The system of claim 1, wherein the computer-executable instructions, when executed, further cause the processor to:
estimate a fact summary based at least in part on the nutrition data associated with the at least one ingredient, wherein the fact summary comprises a name of a recipe, a description of the preparation, delivery, and sourcing of the at least one ingredient, and a list of nutrients associated with the at least one ingredient.

13. The system of claim 1, wherein the computer-executable instructions, when executed, further cause the processor to:
estimate, based at least in part on the nutrition data associated with the at least one ingredient, a nutricise, wherein the nutricise comprises a learning activity that can help users understand healthy eating habits that incorporates physical fitness into the activity.

14. The system of claim 1, wherein the computer-executable instructions, when executed, further cause the processor to estimate an economic impact based on the nutrition data.

15. The system of claim 1, wherein the processor is configured to receive a picture of a food from a user device, and wherein the processor is configured to determine a type and a quantity of the food, and wherein the processor is configured to determine the nutritive score based at least in part on the type and the quantity of the food.

16. A system comprising:
one or more computer-readable storage mediums having program instructions embodied therewith; and
one or more processors configured to execute the program instructions to cause the system to perform a computer-implemented method of:
processing an indication of user data, wherein the user data comprises a food item including at least one recipe having one or more ingredients and a query, wherein the query is associated with at least one ingredient;
generating, based at least in part on the query, nutrition data associated with the at least one ingredient, wherein the nutrition data comprises preparation data, sourcing data, delivery data, and a list of nutrients for the at least one ingredient;
estimating, based at least in part on the nutrition data associated with the least one ingredient, a nutritive score;
comparing the estimated nutritive score to a threshold;
based on the compared nutritive score, generating one or more instructions to cause an indication of the nutritive score on a user device; and
transmitting the generated one or more instructions to the user device.

17. The system of claim 16, wherein the program instructions, when executed, further cause the processor to:
process an indication of a second food item, wherein the second food item includes at least one recipe having one or more ingredients;
generate, based at least in part on the query, a second nutrition data associated with the at least one ingredient of the second food item, wherein the nutrition data comprises preparation data, sourcing data, delivery data, and a list of nutrients for the at least one ingredient of the second food item;
estimate, based at least in part of the nutrition data associated with the at least one ingredient of the second food item, a second nutritive score;
compare the nutritive score and the second nutritive score; and
based on the compared nutritive score and the second nutritive score, transmit one or more instructions to cause an indication of the compared nutritive scores on a user device.

18. The system of claim 17, wherein the user data further comprises a dietary need data, and wherein the program instructions, when executed, further cause the processor to:
estimate, based at least in part on the second nutrition data and the diet need data, a second nutritive score;
based on the diet need data, determine a threshold;
compare the estimated second nutritive score to a threshold;
generate one or more instructions based on the second nutritive score, or the diet need data; and
transmit the generated one or more instructions to further cause an indication of the second nutritive score on a user device.

19. A method of generating and displaying a nutritive score to a user, the method comprising:
processing an indication of user data, wherein the user data comprises a food item including at least one recipe having one or more ingredients and a query, wherein the query is associated with at least one ingredient;
generating, based at least in part on the query, nutrition data associated with the at least one ingredient, wherein the nutrition data comprises preparation data, sourcing data, delivery data, and a list of nutrients for the at least one ingredient;
initializing a nutritive model;
estimating, based at least in part on the nutrition data associated with the least one ingredient and the nutritive model, a nutritive score;
comparing the estimated nutritive score to a threshold;
based on the compared nutritive score, generating one or more instructions to cause an indication of the nutritive score on a user device;
transmitting the generated one or more instructions to the user device; and
displaying the nutritive score on the user device.

20. The method of claim 19, further comprising:
processing an indication of a second food item, wherein the second food item includes at least one recipe having one or more ingredients;
generating, based at least in part on the query, a second nutrition data associated with the at least one ingredient of the second food item, wherein the nutrition data comprises preparation data, sourcing data, delivery data, and a list of nutrients for the at least one ingredient of the second food item;
estimating, based at least in part of the nutrition data associated with the at least one ingredient of the second food item, a second nutritive score;
comparing the nutritive score and the second nutritive score;
based on the compared nutritive score and the second nutritive score, transmitting one or more instructions to cause an indication of the compared nutritive scores on a user device; and
displaying the compared nutritive score on the user device.
